(19) United States
(12) Patent Application Publication
(10) Pub. No.: US 2010/0035254 A1
(43) Pub. Date: Feb. 11, 2010

(54) COMPOSITION AND METHOD FOR NUCLEIC ACID SEQUENCING

(75) Inventor: John G.K. Williams, Lincoln, NE (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pacific Biosciences of California, Inc., Menlo Park, CA (US)

(21) Appl. No.: 12/413,466

(22) Filed: Mar. 27, 2009

Related U.S. Application Data
(63) Continuation-in-part of application No. 10/821,689, filed on Apr. 8, 2004.
(60) Provisional application No. 61/040,108, filed on Mar. 27, 2008.
(60) Provisional application No. 60/461,522, filed on Apr. 8, 2003.
(60) Provisional application No. 60/462,988, filed on Apr. 14, 2003.

(51) Int. Cl. C12Q 1/68; C12N 9/96; C12N 11/00; C40B 40/06

(52) U.S. Cl. 435/6; 435/188; 435/174; 506/16

(57) ABSTRACT

The present invention provides compositions and methods for detecting incorporation of a labeled nucleotide triphosphate onto the growing end of a primer nucleic acid molecule. The method is used, for example, to genotype and sequence a nucleic acid. In a preferred embodiment, the method described herein detects individual NTP molecules.

### CROSS-REFERENCES TO RELATED APPLICATIONS

The present application is a continuation-in-part application of U.S. application Ser. No. 10/821,689 filed Apr. 8, 2004, pending, which application claims priority to U.S. Provisional Patent Nos. 60/461,522 and 60/462,988, filed on Apr. 8, 2003 and Apr. 14, 2003. The present application also claims priority to U.S. Provisional Patent No. 61/040,108, filed Mar. 27, 2008. The foregoing applications are hereby incorporated in their entirety for all purposes.

### STATEMENT AS TO RIGHTS TO INVENTIONS MADE UNDER FEDERALLY SPONSORED RESEARCH OR DEVELOPMENT

The research embodied within the present application was funded in-part by the Federal Government in research grant numbers R44 HG02292 and R44 HG02066. The invention described in this application was also supported in part by the National Institutes of Health (3R44HG002292-04S1, 5P01HG003015-02). The government has certain rights in the invention.

### BACKGROUND OF THE INVENTION

Significant interest in the sequencing of single DNA molecules dates to 1989 when Keller and colleagues began experimenting with “sequencing by degradation.” In their experiments, isolated fully-labeled DNA molecules are degraded by an exonuclease, and individual labeled bases are detected as they are sequentially cleaved from the DNA (Jett, J. H. et al., Journal of biomolecular structure & dynamics, 7, 301-309 (1989); Stephan, J. et al., Journal of biotechnology, 86, 255-267 (2001); Werner, J. H. et al., Journal of biotechnology, 102, 1-14 (2003)). This approach was ultimately compromised by poor DNA solubility caused by the densely-packed dye labels. More recently, alternative single-molecule approaches have been investigated, including “sequencing by synthesis,” where bases are detected one at a time as they are sequentially incorporated into DNA by a polymerase (Braslavsky, I. et al., Proceedings of the National Academy of Sciences of the United States of America, 100, 3960-3964 (2003); Levene, M. J. et al., Science, 299, 682-686 (2003); Metzker, M. L., Genome research, 15, 1767-1776 (2005)); and nanopore sequencing where electrical signals are detected while single DNA molecules pass through protein or solid-state nanopores (Akeson, M. et al., Biophysical journal, 77, 3227-3233 (1999); Lagerqvist, J. et al., Nano letters, 6, 779-782 (2006); Rhee, K. J. et al., Annals of emergency medicine, 13, 916-923 (1984)). So far, only sequencing by synthesis has been successful. In the method of Quake and colleagues (Braslavsky, I. et al., Proceedings of the National Academy of Sciences of the United States of America, 100, 3960-3964 (2003)), base-labeled nucleotide triphosphates (dNTPs) are incorporated into DNA immobilized on a microscope coverglass. Each type of dNTP is applied separately in a fluidics cycle, and incorporated bases are imaged on the surface after washing away the excess of free nucleotides. While the obtained sequence reads are short, high sequencing rates can potentially be achieved by analyzing billions of different, individual molecules in parallel with applications in re-sequencing and gene expression profiling. Other applications such as denovo sequencing or cancer genome sequencing would benefit from longer reads.

To obtain long single-molecule reads, potentially tens of kilobases, sequencing-by-synthesis approaches using phosphate-labeled nucleotides have been developed (Levene, M. J. et al., Science, 299, 682-686 (2003)). These nucleotides are labeled with a fluorophore on the terminal phosphate instead of on the base. Labeled nucleotides are detected while bound to polymerase during the catalytic reaction. The label is released with pyrophosphate as the nucleotide is incorporated into DNA. An advantage is that the DNA remains label-free and fully soluble. Individual polymerase enzymes immobilized on a microscope coverglass would be monitored in real time to detect the sequence of incorporated nucleotides. In order to achieve long reads, the polymerase, but not the DNA, can be attached to the coverglass. Polymerase attachment facilitates detection because it keeps the active site at a single position on the coverglass surface. In the alternative format, with the polymerase in solution and the DNA attached, the enzyme active site would be a moving target for detection, diffusing up to several microns from the DNA attachment point as the primer strand is extended from long templates.

U.S. Pat. No. 6,255,083, issued to Williams and incorporated herein by reference, discloses a single molecule sequencing method on a solid support. The solid support is optionally housed in a flow chamber having an inlet and outlet to allow for renewal of reactants that flow past the immobilized polymerases. The flow chamber can be made of plastic or glass and should either be open or transparent in the plane viewed by the microscope or optical reader.

U.S. Pat. No. 4,979,824, illustrates that single molecule detection can be achieved using flow cytometry wherein flowing samples are passed through a focused laser with a spatial filter used to define a small volume. Moreover, U.S. Pat. No. 4,793,705 describes a detection system for identifying individual molecules in a flow train of the particles in a flow cell. The patent further describes methods of arranging a plurality of lasers, filters and detectors for detecting different fluorescent nucleic acid base-specific labels.

Single molecule detection on a solid support is described in Ishikawa, et al. Jan. J. Apple. Phys. 33:1571-1576. (1994). As described therein, single-molecule detection is accomplished by a laser-induced fluorescence technique with a position-sensitive photon-counting apparatus involving a photon-counting camera system attached to a fluorescence microscope. Laser-induced fluorescence detection of a single molecule in a capillary for detecting single molecules in a quartz capillary tube has also been described. The selection of lasers is dependent on the label and the quality of light required. Diode, helium neon, argon ion, argon-krypton mixed ion, and Nd:YAG lasers are useful in this invention (see, Lee et al. (1994) Anal. Chem., 66:4142-4149).

The predominant method used today to sequence DNA is the Sanger method (Proc. Natl. Acad. Sci. 1977, 74, 5463) which involves use of dideoxynucleoside triphosphates as DNA chain terminators. Most high throughput-sequencing systems use this approach in combination with use of fluorescent dyes. The dyes may be attached to the terminator or be a part of the primer. The former approach is preferred as only the terminated fragments are labeled. Multiplexing energy transfer fluorescent dyes are preferable over the use of single dyes.

U.S. Pat. No. 6,306,607 describes modified nucleotides wherein the nucleotide has a terminally labeled phosphate, which characteristic is useful for single-molecule DNA sequencing in a microchannel. Using 4 different NTPs each labeled with a unique dye, real-time DNA sequencing is possible by detecting the released pyrophosphate having different labels. The cleaved PPi-Dye molecules are detected in isolation without interference from unincorporated NTPs and without illuminating the polymerase-DNA complex.

Despite the advances in U.S. Pat. No. 6,255,083, a need currently exists for more effective and efficient compositions, methods, and systems for nucleic acid sequencing. Specifically, a need exists for improved nucleic acid sequencing compositions and methods to increase processivity. These and further needs are provided by the present invention.

### SUMMARY OF THE INVENTION

The current invention provides compositions and methods to sequence nucleic acid. The compositions and methods allow for increasing the processivity index of polymerases and thus, results in more efficient nucleic acid sequencing. As such, in one aspect, the present invention provides a polymerase-nucleic acid complex, the polymerase-nucleic acid complex comprising: a target nucleic acid and a nucleic acid polymerase, wherein the polymerase has an attachment complex comprising at least one anchor which irreversibly associates the target nucleic acid with the polymerase for increasing the processivity index.

In one embodiment, the polymerase-nucleic acid complex further comprises a primer nucleic acid which complements a region of the target nucleic acid. In another embodiment, the attachment complex comprises at least two anchors. In certain instances, the attachment complex is attached to a support. In certain other instances, the at least two anchors in the attachment complex further comprises a topological tether. In yet certain other instances, the topological tether is an antibody and the at least two anchors are for example, each a histidine tag.

In another embodiment, the attachment complex comprises a topological tether. In certain instances, the topological tether comprises an antibody. In yet another embodiment, the topological tether is attached to the at least one anchor via a complementary binding pair. In a further embodiment, the topological tether is attached to the at least two anchors via at least two complementary binding pairs.

In another embodiment, the at least one anchor comprises an at least one amino acid or an epitope for attachment. In certain instances, the at least one amino acid is selected from the group of a cysteine, a phenylalanine derivative or a histidine. In certain other instances, the histidine is selected from the group of a histidine tag, a histidine patch or a polyhistidine sequence.

In yet another embodiment, the at least one anchor is attached to a support. In certain instances, the at least one anchor entraps the target nucleic acid. In a further embodiment, the target nucleic acid is a circular DNA. In certain instances, the circular DNA is sequenced by strand displacement synthesis.

In another embodiment, the polymerase is a selected from a Family A polymerase and a Family B polymerase. In certain instances, the Family A polymerase is selected from the group of Klenow, Taq, and T7 polyermase. In certain other instances, the Family B polymerase is selected from the group of a Therminator polymerase, phi29, RB-69 and T4 polymerase. In yet another embodiment, the polymerase-nucleic acid complex is an array of polymerase-nucleic acid complexes attached to a support. In certain instances, the plurality of members of the array of polymerase-nucleic acid complexes is randomly attached to the support. In certain other instances, the plurality of members of the array of polymerase-nucleic acid complexes is uniformly attached to the support.

In a further embodiment, the processivity index is at least 0.5. In certain instances, the processivity index is at least 0.8. In certain other instances, the processivity index is 1.

In another aspect, the present invention provides a method for detecting incorporation of at least one NTP into a single primer nucleic acid molecule, the method comprising:
i. immobilizing onto a support a polymerase nucleic acid complex comprising a target nucleic acid, a primer nucleic acid which complements a region of the target nucleic acid, and at least one nucleic acid polymerase;
ii. contacting said immobilized complex with at least one type of labeled nucleotide triphosphate [NTP], wherein each NTP is labeled with a detectable label, and
iii. detecting the incorporation of the at least one type of labeled NTP into a single molecule of the primer, while the at least one type of labeled NTP is in contact with the immobilized complex, by detecting the label of the NTP while the at least one type of labeled NTP is in contact with the polymerase nucleic acid complex.

In one embodiment, the polymerase nucleic acid complex is contacted with a single type of labeled NTP. In another embodiment, the polymerase nucleic acid complex is contacted with at least two different types of NTPs, and wherein each type of NTP is uniquely labeled. In yet another embodiment, the polymerase nucleic acid complex is contacted with at least four different types of NTPs, and wherein each type of NTP is uniquely labeled. In a further embodiment, the NTPs are labeled on the γ-phosphate. In certain instances, the NTPs are labeled on the γ-phosphate with a fluorescent label.

In another embodiment, detecting the incorporation of the at least one type of labeled NTP into a single molecule of the primer comprises detecting a unique signal from the labeled NTP using a system or device selected from the group of an optical reader, a high-efficiency photon detection system, a photodiode, a camera, a charge couple device, an intensified charge couple device, a near-field scanning microscope, a far-field confocal microscope, a microscope that detects wide-field epi-illumination, evanescent wave excitation and a total internal reflection fluorescence microscope. In yet another embodiment, the label of the NTP is detected using a method comprising a four color evanescent wave excitation device. In a further embodiment, detecting the incorporation of the at least one type of labeled NTP into a single molecule of the primer is carried out by a mechanism selected from the group of fluorescence resonance energy transfer, an electron transfer mechanism, an excited-state lifetime mechanism and a ground-state complex quenching mechanism.

In yet another embodiment, detecting the incorporation of the at least one type of labeled NTP into a single molecule of the primer comprises measuring a residence time of a labeled NTP in the polymerase nucleic acid complex. In certain instances, the residence time of an NTP that is incorporated into the primer nucleic acid is at least about 100 times longer to about 10,000 times longer than the residence time of an NTP that is not incorporated. In certain other instances, the residence time of an NTP that is incorporated into the primer nucleic acid is at least about 200 times longer to about 500 times longer than the residence time of an NTP that is not incorporated. In yet certain other instances, the residence time of an NTP that is incorporated into the primer nucleic acid is about 1.0 milliseconds to about 100 milliseconds. In further instances, the residence time of an NTP that is incorporated into the primer nucleic acid is about 2.0 milliseconds to about 10.0 milliseconds.

In another embodiment, the method of the present invention further comprises the step of genotyping the target nucleic acid by determining the identity of at least one NTP that is incorporated into a single molecule of the primer. In yet another embodiment, the method of the present invention further comprises sequencing the target nucleic acid by determining the identity and sequence of incorporation of NTPs that are incorporated into a single molecule of the primer.

In a further embodiment, the detection is a sequential detection of the identities of more than one uniquely labeled dNTPs that are sequentially incorporated into the primer, wherein the sequential detection yields the sequence of region of the target DNA that is downstream of the elongating end of the primer. In another embodiment, the polymerase-nucleic acid complex comprises a target nucleic acid and a nucleic acid polymerase, wherein the polymerase has an attachment complex comprising at least one anchor, which irreversibly associates the target nucleic acid with the polymerase for increasing the processivity index.

In certain embodiments, the present invention provides immobilized polymerases, which preferably utilize phosphate-labeled nucleotides, such as terminal phosphate labeled. In certain aspects, starting with the Therminator™ variant of 9°N DNA polymerase (Gardner, A. F. and Jack, W. E., Nucleic acids research, 30, 605-613 (2002); Southworth, M. W. et al., Proceedings of the National Academy of Sciences of the United States of America, 93, 5281-5285 (1996)), directed evolution is used to improve activity with phosphate-labeled dNTPs 26-fold, from for example, 0.8 nt/sec to 21 nt/sec measured at 74° C.

In certain aspects, the polymerase is attached to a nonstick coverglass surface, oriented to permit access to the DNA and nucleotide substrates while allowing for the normal protein conformational changes associated with the catalytic cycle. In still other aspects, the compositions and methods herein allow for extreme processivity, with the polymerase holding on to the same template DNA molecule for the duration of the sequencing run.

These and other objects and advantages will become more apparent when read with the accompanying detailed description and drawings that follow.

### DESCRIPTION OF THE DRAWINGS

FIG. 1 illustrates various features of a polymerase-nucleic acid complex of the present invention.

FIG. 2 illustrates an anchor embodiment of the present invention.

FIG. 3 illustrates a nucleic acid sample preparation of the present invention.

FIG. 4 illustrates a nucleic acid sample preparation of the present invention.

FIG. 5 illustrates a nucleic acid sample preparation of the present invention.

FIG. 6 illustrates a single molecule isolation embodiment of the present invention.

FIG. 7 illustrates a single molecule bound to a cover slip.

FIG. 8 illustrates a multiple sequencing embodiment of the present invention.

FIG. 9A-C illustrates a synthetic scheme of a compound useful in the present invention.

FIG. 10 illustrates a schematic view of a setup for a residence-time detector.

FIG. 11 illustrates a computer simulation of incorporation events detected above a signal energy threshold of 2500. The experimental parameters are summarized in Table III.

FIG. 12 illustrates a computer simulation of background incorporation using the same experimental parameters (summarized in Table III) used in FIG. 11.

FIG. 13A-B illustrates an embodiment of the present invention.

FIG. 14 illustrates a gel showing purified polymerases with AviTag legs of the present invention.

FIG. 15 illustrates a gel of binary complexes of polymerase and streptavidin resolved by isoelectric focusing.

FIG. 16A-C illustrates ternary complexes made with primed M13 DNA, polymerase (zero, one or two AviTag legs) and Alexa Fluor-680-streptavidin (lanes 3-6), with controls omitting either DNA or polymerase (lanes 1 and 2). Panel A shows the parent enzyme without AviTag legs (P); the two single-leg variants (B53 and B229); and the dual-leg polymerase (DBio). Panel B shows a gel from (A), but stained with SYBR Gold™ to visualize the DNA by UV transillumination (312 nm). Panel C shows a gel of purified complexes of DBio polymerase.

FIG. 17 illustrates thermal stability of ternary complexes. Panel A is labeled the labeled streptavidin component; Panel B shows for each lane, the fluorescence signals co-migrating with the two DNA bands (circular, linear) was summed and the results were plotted normalized to the 20° C. sample. The data points were connected by a piecewise spline curve.

FIG. 18A-B Panel A shows DNA synthesis by ternary complexes, wherein complexes (1.2 nM) made with labeled streptavidin were mixed with 200 μM of each of the 4 unlabeled dNTPs and 5 mM MgCl₂ in buffer C and were incubated at 54° C. for 0, 3, 10, 30 and 90 min (lanes 2-6). In Panel B, the streptavidin component was imaged by fluorescence using a LI-COR Odyssey infrared imager; controls include lanes 1 and 8, and M13 fully extended with a saturating amount of Taq DNA polymerase is lane 7.

FIG. 19A-B Panel A shows polymerization kinetics of purified complexes, wherein DNA synthesis rates (v, nt/sec) were determined to be Km=54 μM, Vmax=3.4 nt/s. Panel B shows the control sample of uncomplexed polymerase and DNA: Km=21 μM, Vmax=12.0 nt/s.

FIG. 20A-C Panel A shows DNA synthesis by immobilized complexes, wherein purified ternary complexes made with unlabeled streptavidin were immobilized in a reaction chamber on a PEG-biotin coated coverglass. Panel B shows the control reaction inhibiting polymerase activity by replacing Mg⁺⁺ with 0.1 mM EDTA. Panel C shows zoomed-in view of a single DNA spot showing movement.

FIG. 21 shows a gel which indicates that complexes are stable to freezing and thawing.

FIG. 22 illustrates on embodiment, wherein site-directed, Ligase-Independent Mutagenesis (SLIM) is used for inserting the AviTag legs.

FIG. 23 illustrates an embodiment of a chamber used in immobilization aspects of the present invention.

### DETAILED DESCRIPTION OF THE INVENTION

### I. Embodiments

In certain aspects, the present invention provides a processivity complex, the processivity complex comprising: a polymerase; and a template nucleic acid, the polymerase and template nucleic acid forming a complex immobilized on a surface.

In certain instances, the present invention provides an artificial processivity complex that both traps the template DNA on the polymerase and facilitates oriented immobilization on biotinylated surfaces. Starting with the parent polymerase adapted to phosphate-labeled dNTPs, in certain embodiments, AviTag™ peptide “legs” are inserted at two surface-exposed locations flanking the DNA binding cleft. The AviTag peptides provide highly specific sites for enzymatic biotinylation of the polymerase by E. coli biotin-protein ligase. Processivity is enhanced with streptavidin binding the AviTag legs, retaining the template in the DNA binding cleft. The template DNA is stably associated with the polymerase, and the polymerase-DNA-streptavidin complexes are active both in solution and when immobilized on biotinylated coverglass surfaces. Advantageously, the clamp converts a naturally non-processive DNA polymerase into a highly-processive one capable of incorporating thousands of nucleotides without dissociating from the template.

### II. Polymerase-Nucleic Acid Complex

In one embodiment, the present invention provides a polymerase-nucleic acid complex (PNAC), comprising: a target nucleic acid and a nucleic acid polymerase, wherein the polymerase has an attachment complex comprising at least one anchor, which at least one anchor irreversibly associates the target nucleic acid with the polymerase to increase the processivity index. As used herein, the term “processivity index” means the number of nucleotides incorporated before the polymerase dissociates from the DNA. Processivity refers to the ability of the enzyme to catalyze many different reactions without releasing its substrate. That is, the number of phosphodiester bonds formed using the present invention is greatly increased as the substrate is associated with polymerase via an anchor.

In one embodiment, the processivity index is defined as the number of nucleotides sequenced divided by the number of nucleotides in the template. For example, if the template is 10,000 bases long, and the PNAC sequences 9000 bases, the index is 0.90. Using the PNACs and methods of the present invention, the index is preferably between at least 0.5 to about 1. More preferably, the index is about at least 0.80 to about 1, such as at least 0.80, or at least 0.85, or at least 0.90, or at least 0.95, or 1.0.

Using the PNACs of the present invention, because the target is irreversibly associated with the polymerase, the number of nucleotides added can be from about 20 to about 100,000, such as about 1000 to about 30,000, such as about 5000 to about 20,000.

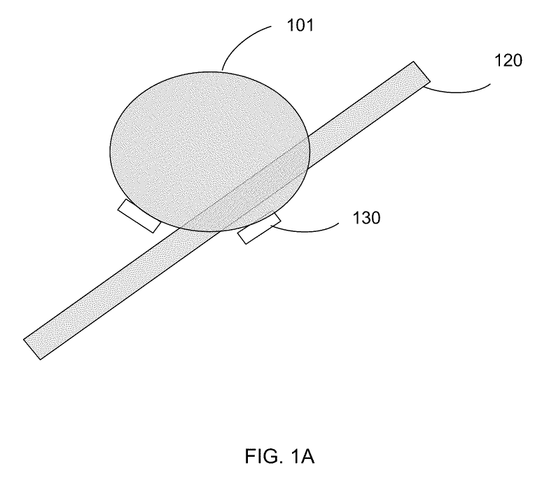

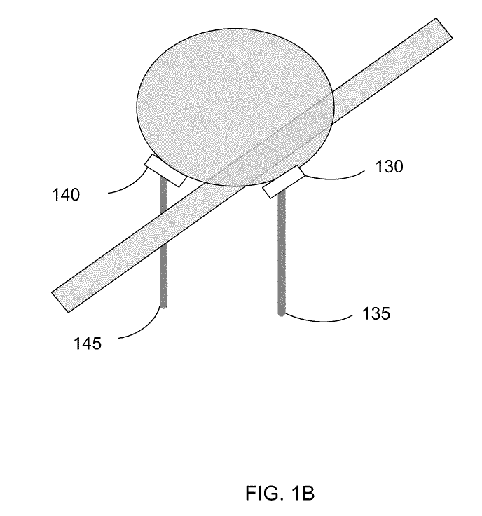

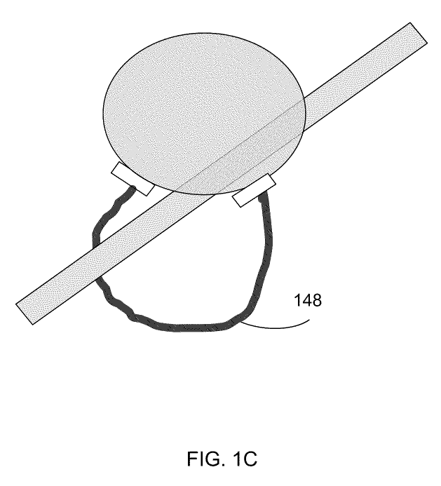

FIG. 1A-D are examples of polymerase nucleic acid complexes (PNACs) of the present invention. This diagram is merely an illustration and should not limit the scope of the claims herein. One of ordinary skill in the art will recognize other variations, modifications, and alternatives.

The polymerase-nucleic complex comprises at least one anchor. In certain aspects, the PNAC will further comprise a primer, which complements a region of the target nucleic acid. As shown in FIG. 1A, the polymerase 101 can have at least one anchor 130 such anchor comprising for example, an amino acid, an epitope, a modified amino acid and the like, for attaching a topological tether. The amino acid i.e., anchor can be for example, a cysteine or a histidine. In certain aspects, the polymerase nucleic acid complex, wherein the nucleic acid 120 is preferably within the active site, comprises at least two anchors. Suitable anchors of the present invention include, but are not limited to, an amino acid, a modified amino acid, a peptide, a histidine tag, a histidine patch, an eptiope, and the like. In certain instances, the at least one anchor entraps the target nucleic acid such as by folding back on itself. In other instances, the anchors of the present invention are useful for also attaching a topological tether to the polymerase, or for example, attaching the PNAC to a substrate. In other embodiments, the anchor affixes the PNAC to a support, with or without a topological tether. In certain other embodiments, the polymerase-nucleic complex comprises a topological tether bound to at least two anchors.

As shown in FIG. 1B, an anchor 130 can further comprise other functionalities such as a first member 135 of a first binding pair. A second anchor 140 has a first member 145 of a second binding pair. As shown in FIG. 1C, in certain instances, a topological tether is formed when the first members 135, 145 are joined by a common member 148. Alternatively, a topological tether can be formed when the first members 135, 145 are each joined directly to a support (not shown). A topological tether and at least one anchor can attach via complementary binding pairs. Alternatively, the anchors can attach directly to a substrate without the use of a tether (for example, histidine patches as anchors bound directed to a Ni surface). Suitable complementary binding pairs include, but are not limited to, any haptenic or antigenic compound in combination with a corresponding antibody or binding portion or fragment thereof, nonimmunological binding pairs, receptor-receptor agonist or antagonist, IgG-protein A, lectin-carbohydrate, enzyme-enzyme cofactor, enzyme-enzyme-inhibitor, and complementary polynucleotide pairs capable of forming nucleic acid duplexes.

Exemplary complementary binding pairs include, but are not limited to, digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin, biotin-avidin, biotin-streptavidin, thyroxine and cortisol, histidine patch and Ni-NTA and acetylcholine and receptor-acetylcholine. In certain aspects, the anchor comprises at least one amino acid or an epitope for attaching the topological tether.

As discussed, in certain instances, anchors can comprise an amino acids capable of modification for attachment to a binding member, a tether, a support, and combinations thereof. In one embodiment, a topological tether can attach to two anchors, without intervening binding pairs.

In one aspect, the anchor comprises a biotin moiety. For example, biotin-X nitrilotriacetic acid can be used to covalently attach the biotin moiety to a protein having a free amino group. In turn, this biotin anchor can attach to a streptavidin or a neutraviden binding member, or alternatively, directly to a streptavidin or a neutravidin support.

In another aspect, the topological tether comprises an antibody. In certain embodiments, the topological tether is an antibody that can attach via anchors having complementary binding pairs. For example, the two anchors can be histidine tags, and the tether can be an antibody. In certain aspects, the polymerase-nucleic complex comprises a topological tether anchored to a solid support 150 (see, FIG. 1D).

In certain aspects, the polymerase-nucleic acid attachment complex can be attached to the substrate by providing an anchor such as a polyhistidine tag, that binds to metal. Other conventional means for attachment employ binding pairs. Alternatively, covalent crosslinking agents can be employed such as reagents capable of forming disulfide (S—S), glycol (—CH(OH)—CH(OH)—), azo (—N═N—), sulfone (—S(═O2-), ester (—C(═O)—O—), or amide (—C(═O)—N—) bridges. The covalent bond is for example, an amide, a secondary or tertiary amine, a carbamate, an ester, an ether, an oxime, a phosphate ester, a sulfonamide, a thioether, a thiourea, or a urea.

Selected examples of reactive functionalities useful for the attaching an anchor to the polymerase, a tether to the anchor, or the PNAC to the substrate are shown in Table I, wherein the bond results from such a reaction. Those of skill in the art will know of other bonds suitable for use in the present invention.

TABLE I

| Reactive functionality | Complementary group | The resulting bond |
|---|---|---|
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides/ |
| | | imides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or |
| | | anhydrides |
| diazoalkanes | carboxylic acids | esters |
| epoxides | thiols (amines) | thioethers |
| | | (alkyl amines) |
| epoxides | carboxylic acids | esters |
| haloacetamides | thiols | thioethers |
| haloplatinate | amino | platinum complex |
| haloplatinate | heterocycle | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonyl halides | amines/anilines | sulfonamides |

In certain aspects, the polymerase can be covalently attached to a support (e.g., coverslip, metal surface, and the like), wherein the polymerase is labeled in vivo with a modified amino acid such as for example, a benzaldehyde derivative of phenylalanine. In one example, the benzaldehyde derivative of phenylalanine is p-acetyl-L-phenylalanine, which can be labeled at specific position(s) in the polymerase. This can be accomplished using organisms (e.g., E. coli, yeast) engineered to have an augmented 21-amino acid genetic code capable of inserting p-acetyl-L-phenylalanine at specific codons (see, Lei Wang, Zhiwen Zhang, Ansgar Brock, Peter G. Schultz (2003) Proc Natl Acad Sci USA 100:56-61). In one aspect, the polymerase gene of the present invention is engineered to have the appropriate codon or codons at the desired anchor positions, and the corresponding polymerase protein is expressed in the 21-amino acid organism. The expressed polymerase is then purified, mixed with the template DNA, and the resulting PNACs are contacted to a support derivatized with a hydrazine, hydrazone, and the like (e.g., SANH from Solulink Inc). Alternatively, a chemical functionality equivalent to p-acetyl-L-phenylalanine can be attached to the protein at specific or unspecific positions by conjugating SFB (Solulink Inc) to lysine amino acids on the protein. The functionalized protein is attached to the support as above.

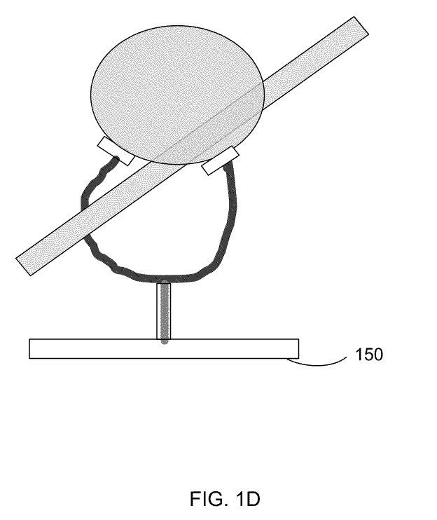

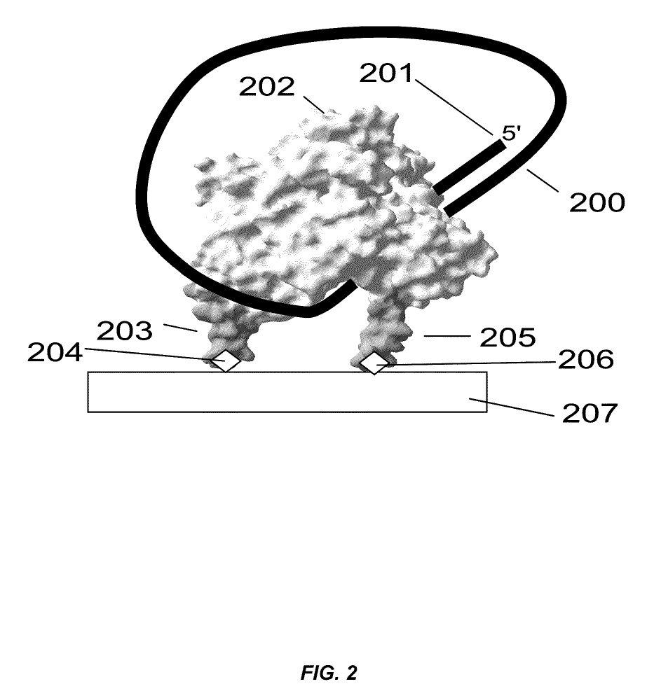

FIG. 2 shows a structural model of a PNAC comprising a 9 Degrees North DNA polymerase (parent of Therminator polymerase) 202 and a circular primed DNA template 200. This diagram is merely an illustration and should not limit the scope of the claims herein. One of ordinary skill in the art will recognize other variations, modifications, and alternatives. The polymerase 202 comprises anchors 203 and 205 inserted at Therminator amino acid positions K53 and K229, respectively. The anchors are identical in amino acid sequence (LLSKKRSLCCXCTVIVYVTDT), wherein the anchor comprises amino acid pa-Phe, which is indicated by “X” in the sequence and by white diamonds 204, 206. The pa-Phe amino acids 204, 206 are shown attached to the support 207. The circular DNA template 200 is hybridized to a primer 201. The 5′-end of the primer is indicated 201 and the 3′-end of the primer is hidden in the DNA binding cleft of the protein 202. The structural model is 1QHT.pdb in the protein database at http://www.rcsb.org/pdb/.

As discussed, the Therminator DNA polymerase can be modified by inserting a 20-amino acid anchor at position K53 and a 20-amino acid anchor at position K229 in the Therminator gene. These two positions straddle the DNA binding cleft as shown in FIG. 2. As shown therein, each 20-amino acid anchor is engineered to contain at least one p-acetyl-L-phenylalanine (pa-Phe) amino acid near the middle of the anchor (FIG. 2). The engineered protein is then purified. In one embodiment, to make polymerase nucleic acid complexes, the purified Therminator protein is mixed with a primed single stranded circular DNA template and the mixture is contacted with a support derivatized with hydrazine or hydrazone linkers (Solulink Inc). Optionally, the template DNA contains at least one dUTP base positioned 4-5 bases from the 3′-end of the primer in order to stabilize the polymerase-DNA complex as described (see, Mark Fogg, Laurence Pearl, Bernard Connolly (2002) Nature Structural Biology 9:922-927). The polymerase-DNA complex attaches to the support by bond formation between the pa-Phe on the protein and the hydrazine or hydrazone linker on the support. Optionally, the kinetics of bond formation can be increased by concentrating polymerase-DNA complexes on the support surface using an energy field (e.g., electric field, pressure field, magnetic field, and the like). Once the PNAC has formed on the support, the circular DNA is irreversibly associated with the polymerase as shown in FIG. 2.

A. Polymerases

The polymerases suitable for use in the present invention preferably have a fidelity (incorporation accuracy) of at least 99%. In addition, the processivity of the polymerase should be at least 20 nucleotides, prior to immobilization. Although the polymerase selected for use in this invention is not critical, preferred polymerases are able to tolerate labels on the γ-phosphate of the NTP.

In certain aspects, the polymerases useful in the present invention are selected from the A family polymerases or the B family polymerases. DNA-dependent DNA polymerases have been grouped into families, including A, B, X, and others on the basis of sequence similarities. Members of family A, which includes bacterial and bacteriophage polymerases, share significant similarity to E.coli polymerase I; hence family A is also known as the pol I family. The bacterial polymerases also contain an exonuclease activity, which is coded for in the N-terminal portion. Family A polymerases include for example, Klenow, Taq, and T7 polymerases. Family B polymerases include for example, the Therminator polymerase, phi29, RB-69 and T4 polymerases.

In certain instances, suitable DNA polymerases can be modified for use in the present invention. These polymerases include, but are not limited to, DNA polymerases from organisms such as Thermus flavus, Pyrococcus furiosus, Thermotoga neapolitana, Thermococcus litoralis, Sulfolobus solfataricus, Thermatoga maritima, E. coli phage T5, and E. coli phage T4. The DNA polymerases may be thermostable or not thermostable.

In other embodiments, the polymerases include T7 DNA polymerase, T5 DNA polymerase, HIV reverse transcriptase, E. coli DNA pol I, T4 DNA polymerase, T7 RNA polymerase, Taq DNA polymerase and E. coli RNA polymerase. In certain instances, exonuclease-defective versions of these polymerases are preferred. The efficiency with which γ-labeled NTPs are incorporated may vary between polymerases; HIV-1 RT and E. coli RNA polymerase reportedly readily incorporate γ-labeled nucleotide. The polymerase can also be a T7 polymerase. T7 polymerase has a known 3D structure and is known to be processive. In order to operate in a strand-displacement mode, the polymerase requires a complex of three proteins: T7 polymerase+thioredoxin+primase (Chowdhury et al. PNAS 97: 12469). In other embodiments, the polymerases can also be HIV RT and DNA Polymerase I.

B. Sources of Target Nucleic Acid.

The identity and source of the template and primer nucleic acid (“NA”) is generally not critical, although particular NAs are needed for specific applications. NA used in the present invention can be isolated from natural sources, obtained from such sources such as ATCC, GenBank libraries or commercial vendors, or prepared by synthetic methods. It can be mRNA, ribosomal RNA, genomic DNA or cDNA, an oligonucleotide, which can be either isolated from a natural source or synthesized by known methods. When the target (i.e., template) NA is from a biological source, there are a variety of known procedures for extracting nucleic acid and optionally amplified to a concentration convenient for genotyping or sequence work. Nucleic acid can be obtained from any living cell of a person, animal or plant. Humans, pathogenic microbes and viruses are particularly interesting sources.

Nucleic acid amplification methods are also known and can be used to generate nucleic acid templates for sequencing. Preferably, the amplification is carried out by polymerase chain reaction (PCR) (U.S. Pat. Nos. 4,683,202. 4,683,195 and 4,889,818; Gyllenstein et al., 1988, Proc. Natl. Acad. Sci. USA 85: 7652-7656; Ochman et al., 1988, Genetics 120: 621-623; Loh et al., 1989, Science 243: 217-220; Innis et al, 1990, PCR PROTOCOLS, Academic Press, Inc., San Diego, Calif.). Other amplification methods known in the art can be used, including but not limited to ligase chain reaction, use of Q-beta replicase, or methods listed in Kricka et al., 1995, MOLECULAR PROBING, BLOTTING, AND SEQUENCING, Chap. 1 and Table IX, Academic Press, New York.

Any NA used in the invention can also be synthesized by a variety of solution or solid phase methods. Detailed descriptions of the procedures for solid phase synthesis of nucleic acids by phosphite-triester, phosphotriester, and H-phosphonate chemistries are widely available. See, for example, Itakura, U.S. Pat. No. 4,401,796; Caruthers, et al., U.S. Pat. Nos. 4,458,066 and 4,500,707; Beaucage, et al., Tetrahedron Lett., 22:1859-1862 (1981); Matteucci, et al., J. Am. Chem. Soc., 103:3185-3191 (1981); Caruthers, et al., Genetic Engineering, 4:1-17 (1982); Jones, chapter 2, Atkinson, et al., chapter 3, and Sproat, et al., chapter 4, in Oligonucleotide Synthesis: A Practical Approach, Gait (ed.), IRL Press, Washington D.C. (1984); Froehler, et al., Tetrahedron Lett., 27:469-472 (1986); Froehler, et al., Nucleic Acids Res., 14:5399-5407 (1986); Sinha, et al. Tetrahedron Lett., 24:5843-5846 (1983); and Sinha, et al., Nucl. Acids Res., 12:4539-4557 (1984) which are incorporated herein by reference.

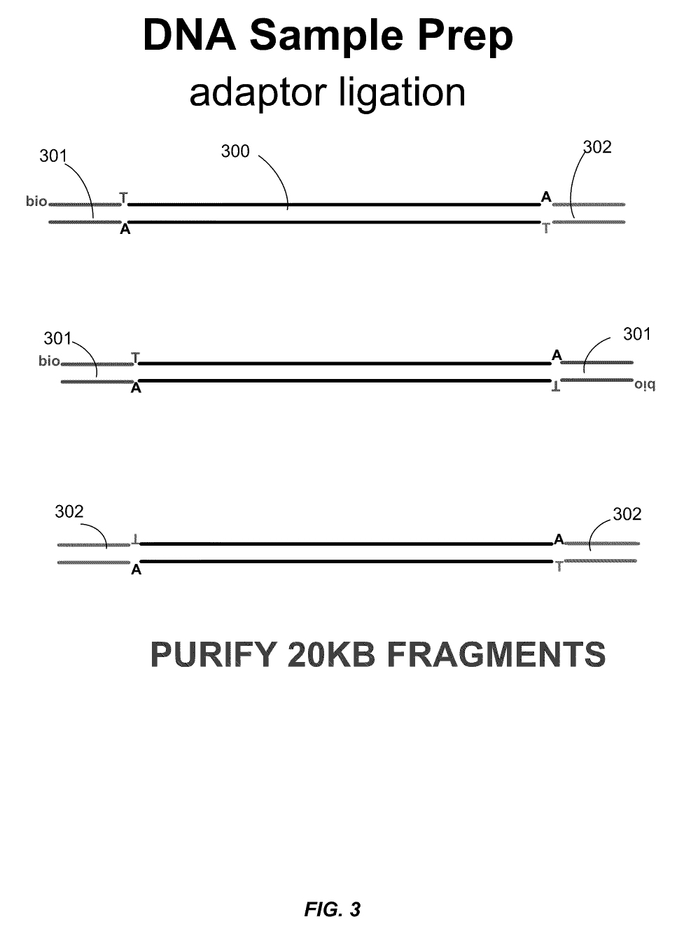

In one preferred embodiment, the target nucleic acid is circular DNA. In one aspect, the circular DNA is sequenced by strand displacement synthesis. As is shown in FIG. 3, randomly-sheared fragments of genomic DNA are purified from a sample organism. The DNA 300 is then treated with for example, T4 DNA polymerase, to generate blunt ends and a single “A” nucleotide is added to the 3′-ends with for example, Taq DNA polymerase, and dATP. A mixture of two double-stranded oligonucleotide adaptors 301 and 302 (each with a “T” nucleotide on one 3′-end to complement the “A” nucleotide on the randomly-sheared fragment) is ligated to the DNA fragments 300 with T4 DNA ligase, wherein the first adaptor 301 is 5′-biotinylated on one strand and the second adaptor 302 is not biotinylated. Whereas the adaptors attach with equal probability to the DNA fragment ends, about half of the ligated DNA molecules will have one biotinylated adaptor and one non-biotinylated adaptor, one quarter will have two biotinylated adaptors, and one quarter will have two non-biotinylated adaptors as shown in FIG. 3. The desired ligated DNA fragment types, having one biotinylated and one non-biotinylated adaptor, are purified after ligation using gel electrophoresis and streptavidin-coated magnetic beads as follows.

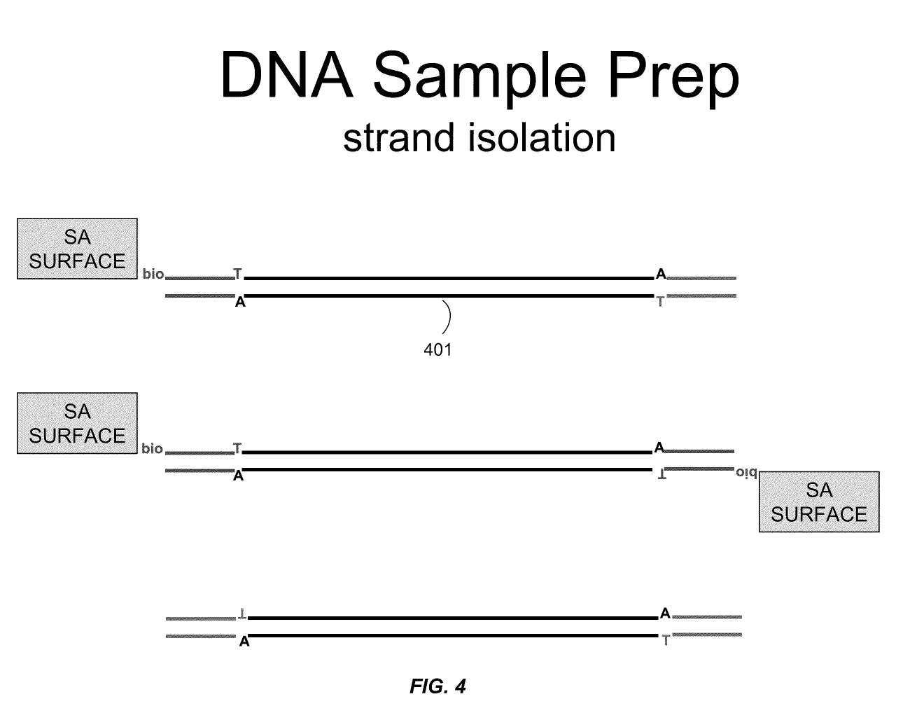

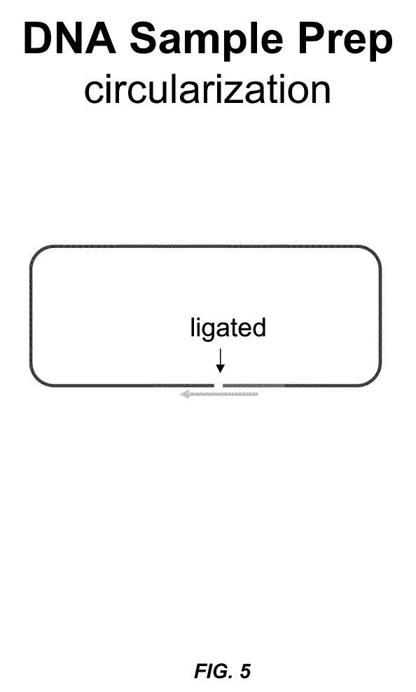

After ligation, DNA fragments in the size range of about 17-23 kb are purified by gel electrophoresis. As shown in FIG. 4, the purified fragments are bound to streptavidin-coated magnetic beads (Dynal). After binding, the beads are washed to remove unbound DNA. Then the bound DNA is denatured at alkaline pH and the unbiotinlyated strands 401 are eluted and the DNA still bound to the beads is discarded. As shown in FIG. 5, the eluted strands are circularized by hybridizing to a primer oligonucleotide complementary to both adaptors and ligating the two ends of the eluted strand.

C. Immobilization of the PNACs

In certain embodiments, the PNAC arrays of the present invention are immobilized on a support. Preferably, the support (e.g., solid support) comprises a bioreactive moiety or bioadhesive layer. The support can be for example, glass, silica, plastic or any other conventionally material that will not create significant noise or background for the detection methods. The bioadhesive layer can be an ionic adsorbent material such as gold, nickel, or copper, protein-adsorbing plastics such as polystyrene (U.S. Pat. No. 5,858,801), or a covalent reactant such as a thiol group.

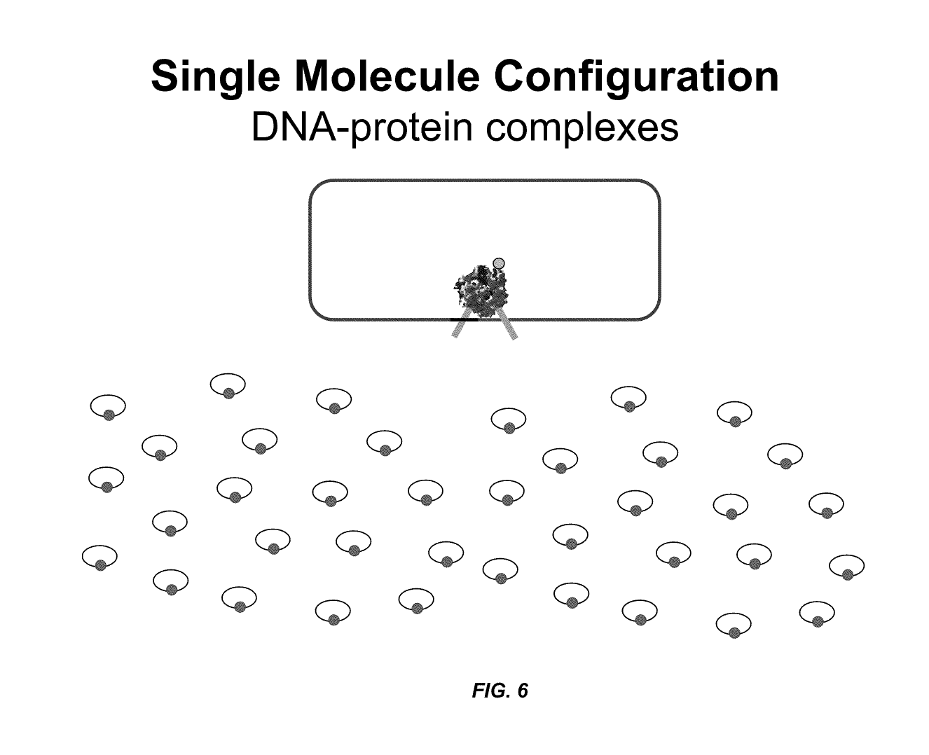

The PNAC arrays of the present invention can be immobilized on a support in a random fashion (e.g., random X or Y position coordinates), uniform fashion (e.g., regularly spaced X or Y position coordinates) or a combination thereof. As is shown in FIG. 6, in one aspect, the PNAC are isolated into single molecule configuration. This single molecule isolation enables efficient attachment of the PNACs to the support. In addition, it allows for efficient single molecule sequencing. Advantageously, the present invention provides single PNACs attached so as to be optically resolvable from their nearest neighbor PNACs. Thus, the PNACs can be analyzed individually without interference from overlapping optical signals from neighboring PNACs. In the present invention, many individual optically resolved PNACs can be sequenced simultaneously.

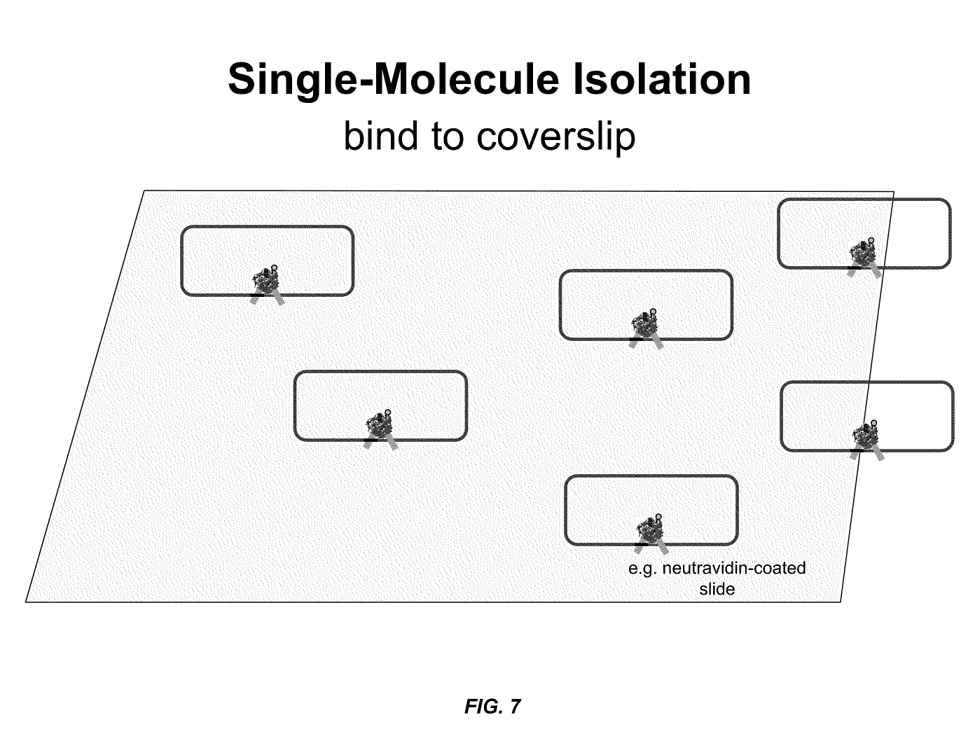

FIG. 7 is an example of a randomly associated array of PNACs immobilized on a neutravidin-coated slide. This diagram is merely an illustration and should not limit the scope of the claims herein. One of ordinary skill in the art will recognize other variations, modifications, and alternatives. As shown therein, PNACs are attached or immobilized to a neutravidin-coated slide via an anchor having for example, the first member of a binding pair, wherein the anchor comprises a biotin moiety. In operation, multiple sites can be sequenced with ease.

In yet another example, the PNACs can be attached to the bioadhesive pattern by providing a polyhistidine tag on the polymerase that binds to metal bioadhesive patterns. To create a patterned or random array of a bioadhesive layer, an electron-sensitive polymer such as polymethyl methacrylate (PMMA) coated onto the support is etched in any desired pattern using an electron beam followed by development to remove the sensitized polymer. The holes in the polymer are then coated with a metal such as nickel, and the polymer is removed with a solvent, leaving a pattern of metal posts on the substrate. This method of electron beam lithography provides the very high spatial resolution and small feature size required to immobilize just one molecule at each point in the patterned array. An alternate means for creating high-resolution patterned arrays is atomic force microscopy. A third means is X-ray lithography.

Other conventional means for attachment employ homobifunctional and heterobifunctional crosslinking reagents. Homobifunctional reagents carry two identical functional groups, whereas heterobifunctional reagents contain two dissimilar functional groups to link the biologics to the bioadhesive. A vast majority of the heterobifunctional cross-linking agents contain a primary amine-reactive group and a thiol-reactive group. Covalent crosslinking agents are selected from reagents capable of forming disulfide (S—S), glycol (—CH(OH)—CH(OH)—), azo (—N═N—), sulfone (—S(═O₂—), ester (—C(═O)—O—), or amide (—C(═O)—N—) bridges.

A bioresist layer may be placed or superimposed upon the bioadhesive layer either before or after attachment of the biologic to the bioadhesive layer. The bioresist layer is any material that does not bind the biologic. Examples include bovine serum albumin, neutravidin, gelatin, lysozyme, octoxynol, polysorbate 20 (polyethenesorbitan monolaurate) and polyethylene oxide containing block copolymers and surfactants (U.S. Pat. No. 5,858,801). Deposition of the layers is done by conventional means, including spraying, immersion and evaporative deposition (metals).

### III. Methods

The present invention provides inter alia, methods to detect incorporation of a detectably labeled nucleotide triphosphate (“NTP”) onto the growing end of a primer nucleic acid molecule. The method is used, for example, to genotype and sequence a nucleic acid. In turn, the sequence identification can be used to identify metabolic differences in patient groups based upon genetic polymorphism to provide improved dosing regimens, enhancing drug efficacy and safety. Further, understanding the genetic basis of disease in animal and plants will help engineer disease resistant animals & crops as well as enhance desirable characteristics.

In a preferred embodiment, the methods described herein detect the “residence time” of an individual fluorogenic NTP molecule on a PNAC preferably comprised of at least one RNA or DNA dependent polymerase, a single target nucleic acid template, and a single primer nucleic acid. The NTPs are preferably labeled with a fluorescent dye, which is preferably attached to the γ-phosphate. As shown in FIG. 8, as the polymerase moves along the target nucleic acid, the nucleotide sequence is read by identifying the order and identity of incorporated NTPs. In one embodiment, all the NTPs have the same label, but each class of labeled NTPs is sequentially added to the complex; the incorporated NTP corresponds to the particular class that is being infused.

In another embodiment, at least two classes of NTP are used, or at least three classes of NTP are used, or at least four classes of NTP are used each of which is uniquely labeled. The identity of the NTP incorporated during a particular incorporation event is determined by detecting the unique label of the incorporated NTP, based on the residence time or the time-averaged intensity of the labeled NTP in contact with the PNAC.

The NTPs can optionally include a fluorescence quencher attached to either the base sugar, dye, polymerase, or combinations thereof, which quenches the fluorescence of the fluorescent dye while the NTP (γ-label) is free in solution. The fluorescence associated with the immobilized complex is detected. Upon interaction with the complex, the fluorescence of the labeled NTP changes (e.g., increases), as the conformation of the NTP is altered by interaction with the complex, and/or as the PPi is cleaved prior to being released into the medium. The optical properties of the pyrophosphate-dye moiety change, either by conformational changes of the NTP or cleavage of the PPi, which in turn facilitates detection of the fluorescent dye.

A. Labeling of NTPs

1. Attachment of a γ-Phosphate Fluorophore

The methods of the present invention involve detecting and identifying individual detectably labeled NTP molecules as a polymerase incorporates them into a single nucleic acid molecule. Suitable nucleobases include, but are not limited to, adenine, guanine, cytosine, uracil, thymine, deazaadenine and deazaguanosine. In certain preferred embodiments, a fluorophore is attached to the γ-phosphate of the NTP by known methods.

The fluorophore may be any known fluorophore including, but not limited to, the following:

TABLE II

| FLUOROPHORE | Absorbance/Emission |
|---|---|
| Rho123 | 507/529 |
| R6G | 528/551 |
| BODIPY 576/589 | 576/589 |
| BODIPY TR | 588/616 |
| Nile Blue | 627/660 |
| BODIPY 650/665 | 650/665 |
| Sulfo-IRD700 | 680/705 |
| NN382 | 778/806 |
| Tetramethylrhodamine | 550 |
| Rodamine X | 575 |
| Cy3 TM | 550 |
| Cy5 TM | 650 |
| Cy7 TM | 750 |

There is a great deal of practical guidance available in the literature for providing an exhaustive list of fluorescent and chromogenic molecules and their relevant optical properties (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949); and the like. Further, there is extensive guidance in the literature for derivatizing fluorophore and quencher molecules for covalent attachment via common reactive groups that can be added to a nucleotide, as exemplified by the following references: Haugland (supra); Ullman et al, U.S. Pat. No. 3,996,345; Khanna et al., U.S. Pat. No. 4,351,760.

There are many linking moieties and methodologies for attaching fluorophore or quencher moieties to nucleotides, as exemplified by the following references: Eckstein, editor, OLIGONUCLEOTIDES AND ANALOGUES: A PRACTICAL APPROACH (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993); Fung et al., U.S. Pat. No. 4,757,141 (5′ phosphoamino group via Aminolink™. II available from Applied Biosystems, Foster City, Calif.); Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

In general, nucleoside labeling can be accomplished using any of a large number of known nucleoside labeling techniques using known linkages, linking groups, and associated complementary functionalities. The linkage linking the quencher moiety and nucleoside should be compatible with relevant polymerases and not quench the fluorescence of the fluorophore moiety.

Suitable dyes operating on the principle of fluorescence energy transfer (FET) include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120),7-amino-4-trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolla Blue; phthalo cyanine; and naphthalo cyanine.

In certain embodiments, certain visible and near IR dyes are known to be sufficiently fluorescent and photostable to be detected as single molecules. In this aspect the visible dye, BODIPY R6G (525/545), and a larger dye, LI-COR's near-infrared dye, IRD-38 (780/810) can be detected with single-molecule sensitivity and are used to practice the present invention.

2. Exemplary Labeled Nucleotides

(i) dATP-PEG-TAMRA

(a) Deprotection of BOC-PEG8-Amine (2)

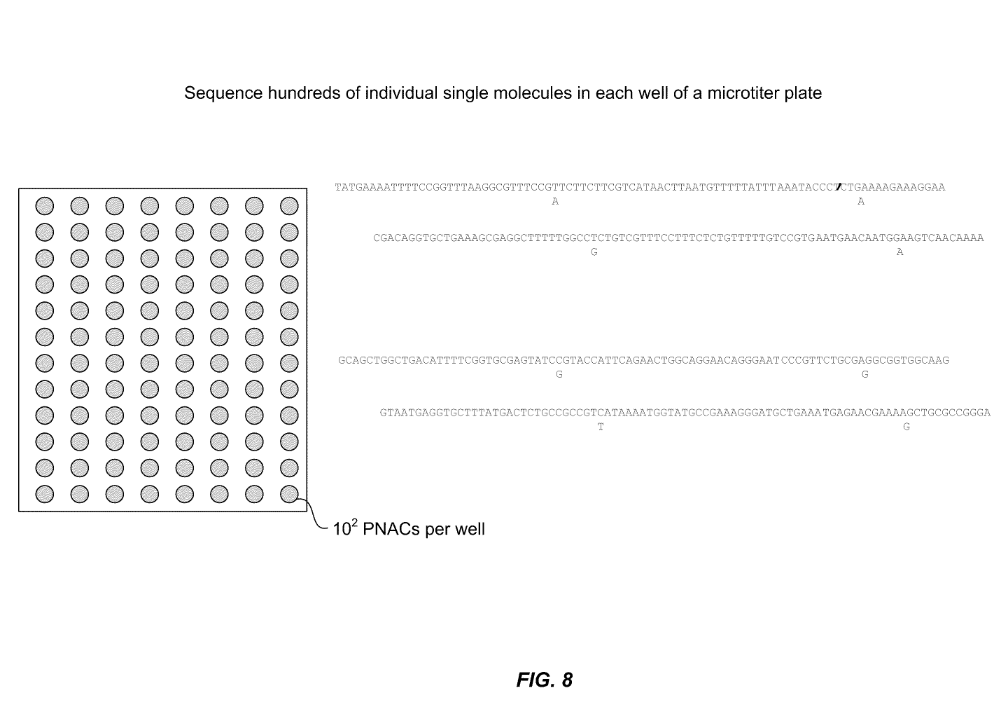

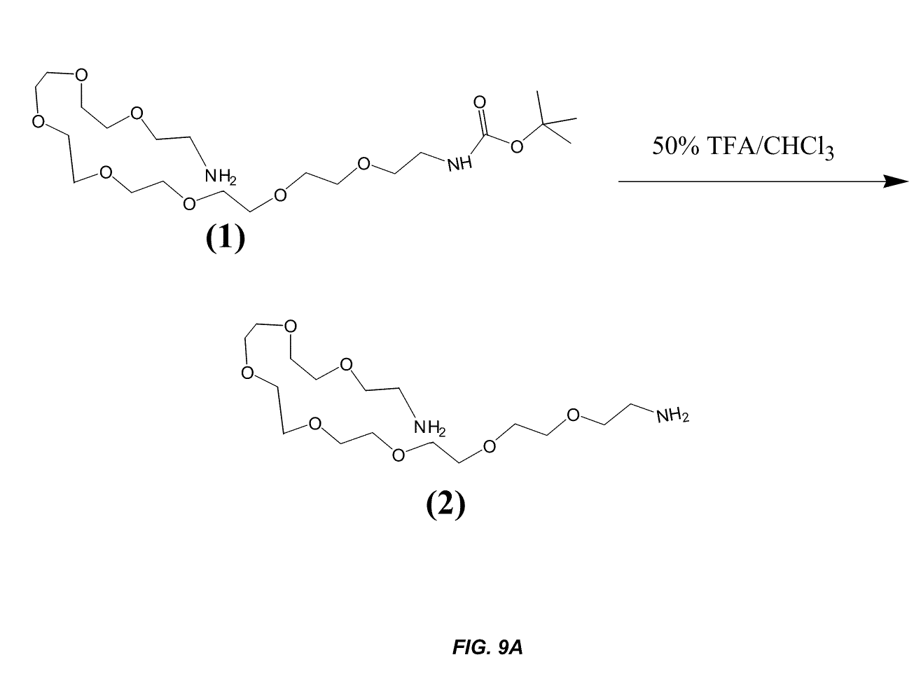

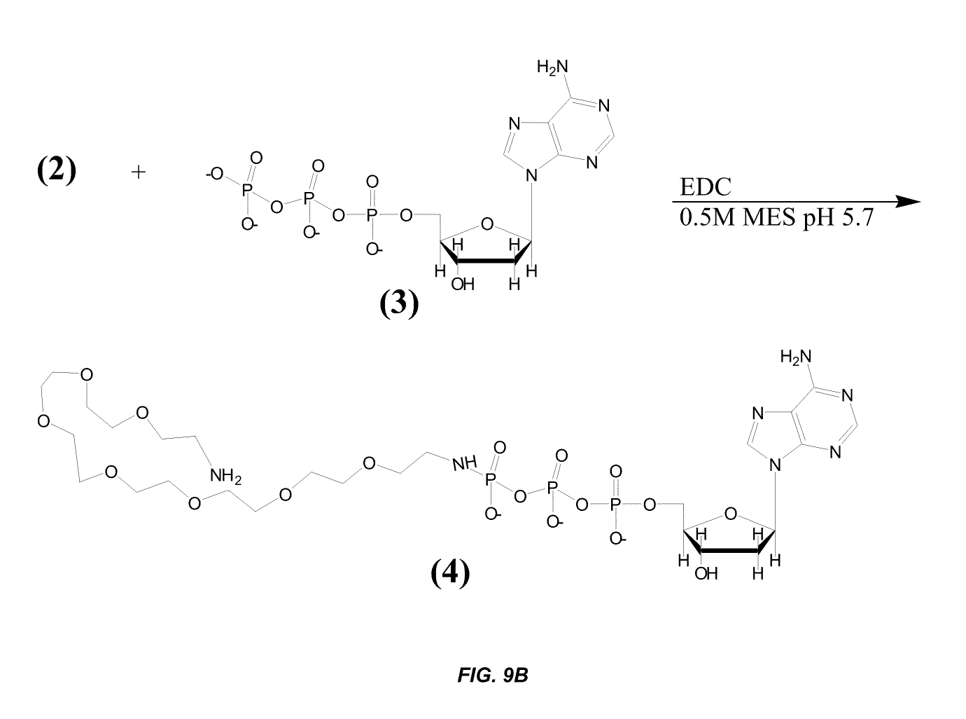

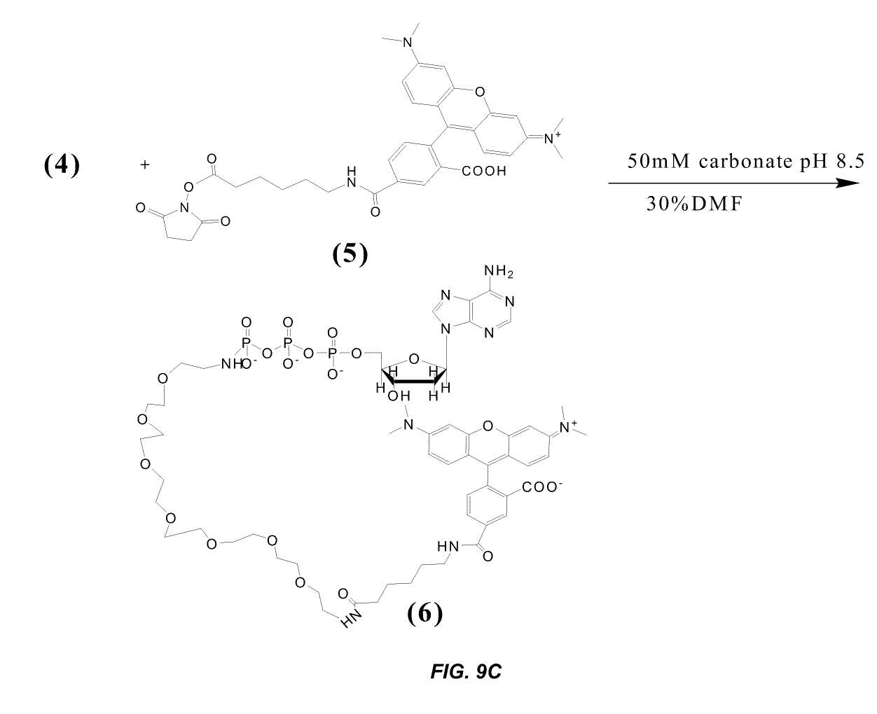

Turning now to FIG. 9A, BOC-PEG8-amine (1) (1g), purchased from PolyPure, is added to a 50% trifluoroacetic acid/chloroform solution (20 mL). The mixture is stirred at room temperature for several hours, and then concentrated down in vacuo to a light orange viscous lquid.

(b) Gamma Labeled dATP (4) With PEG-Diamine (2)

With respect to FIG. 9B, dATP (3) (1 eq., 6.3×10⁻³ mmol, 3.4 mg, 79 mM; Sigma) and EDC (2.5×10⁻¹ mmol, 48.8 mg, 6.5M; Aldrich) are added together in 500 mM MES at pH 5.8. The mixture is allowed to react at room temperature for 10 min. and is then added to the PEG-diamine solution (2) (10 eq., 6.3×10⁻² mmol, 37.5 mg, 31 mM). The pH is adjusted to 5.8-6 using 5M KOH before adding to the nucleotide. The mixture is allowed to react at room temperature for a minimum of 3 hours. The product is first purified on a HiPrep DEAE column (Amersham) using buffer A (10 mM phosphate+20% ACN) and buffer B (Buffer A in 1M NaCl) by holding in buffer A for 10 min and then applying a 0-100% buffer B gradient for 5 minutes. The free PEG is eluted from the column, and then the nucleotide is eluted and collected. A second purification is performed on an Inerstil 10 μm C18 column using buffer A (100 mM TEAAc, pH 6.6-6.8, 4% ACN) and buffer B (100 mM TEAAc, pH 6.6-6.8, 80% CAN) over a period of 15 min. The product is dried in vacuo.

(c) dATP-PEG-TAMRA (6)

With respect to FIG. 9C, the dATP-PEG-amine (4) product is reconstituted in water and quantitated using UV-VIS. dATP-PEG-amine (9.5×10⁻⁵ mmol, 5 μl, 1 eq.), 29 μl in 50 mM carbonate buffer, pH 8, and TAMRA-X SE (5) (1.5eq., 1.4×10⁻⁴ mmol, 9 μl of stock dye solution dissolved at a concentration of 10 mg/mL in DMF; Molecular Probes) are added together. The reaction proceeds at room temperature for 2 hrs. in the dark. Purification of the product is carried out using a HiPrep DEAE column (Amersham) with buffer A (10 mM phosphate+20% ACN) and buffer B (buffer A in 1M NaCl) by holding in buffer A for 10 min and then applying a 0-100% buffer B gradient for 5 minutes. The product is eluted in the void volume. The fractions are collected and concentrated. A second purification step is performed using an Inertsil C18 column with buffer A (100 mM TEAAc, pH 6.6-6.8, 4% ACN) and buffer B (100 mM TEAAc, pH 6.6-6.8, 80%) by applying a 20-100% buffer B gradient over a period of 15 min. The product is dried in vacuo.

In some embodiments of the present invention, detection of pyrophosphate may involve dequenching, or turning on, a quenched fluorescent dye. Efficient quenching lowers background fluorescence, thus enhancing the signal (unquenched NTP fluorescence)-to-noise (quenched NTP fluorescence) ratio. Incomplete quenching results in a low level fluorescence background from each dye molecule. Additional background fluorescence is contributed by a few of the dye molecules that are fully fluorescent because of accidental (i.e., pyrophosphate-independent) dequenching, for example by breakage of a bond connecting the dye to the quencher moiety. Thus, the background fluorescence has two components: a low-level fluorescence from all dye molecules, referred to herein as “distributed fluorescence background” and full-strength fluorescence from a few molecules, referred to herein as “localized fluorescence background.”

In instances where a multi-labeling scheme is utilized, a wavelength which approximates the mean of the various candidate labels' absorption maxima may be used. Alternatively, multiple excitations may be performed, each using a wavelength corresponding to the absorption maximum of a specific label. Table II lists examples of various types of fluorophores and their corresponding absorption maxima.

B. Miscellaneous Reaction Reagents.

The primers (DNA polymerase) or promoters (RNA polymerase) are synthetically made using conventional nucleic acid synthesis technology. The complementary strands of the probes are conveniently synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, e.g. disclosed in the following references: Beaucage and Iyer, Tetrahedron, 48: 2223-2311 (1992); Molko et al, U.S. Pat. No. 4,980,460; Koster et al., U.S. Pat. No. 4,725,677; Caruthers et al., U.S. Pat. Nos. 4,415,732; 4,458,066; and 4,973,679; and the like. Alternative chemistries, e.g. resulting in non-natural backbone groups, such as phosphorothioate, phosphoramidate, and the like, may also be employed provided that the resulting oligonucleotides are compatible with the polymerase. They can be ordered commercially from a variety of companies which specialize in custom oligonucleotides.

Primers in combination with polymerases are used to sequence target DNA. Primer length is selected to provide for hybridization to complementary template DNA. The primers will generally be at least 10 bp in length, usually at least between 15 and 30 bp in length. Primers are designed to hybridize to known internal sites on the subject target DNA. Alternatively, the primers can bind to synthetic oligonucleotide adaptors joined to the ends of target DNA by a ligase. Similarly where promoters are used, they can be internal to the target DNA or ligated as adaptors to the ends.

C. Reaction Conditions.

The reaction mixture for the sequencing using the PNACs and methods of the present invention comprises an aqueous buffer medium which is optimized for the particular polymerase. In general, the buffer includes a source of monovalent ions, a source of divalent cations and a buffering agent. Any convenient source of monovalent ions, such as KCl, K-acetate, NH₄-acetate, K-glutamate, NH₄Cl, ammonium sulfate, and the like may be employed, where the amount of monovalent ion source present in the buffer will typically be present in an amount sufficient to provide for a conductivity in a range from about 500 to 20,000, usually from about 1000 to 10,000, and more usually from about 3,000 to 6,000 microhms.

The divalent cation may be magnesium, manganese, zinc and the like, where the cation will typically be magnesium. Any convenient source of magnesium cation may be employed, including MgCl₂, Mg-acetate, and the like. The amount of Mg ion present in the buffer may range from 0.5 to 20 mM, but will preferably range from about 1 to 12 mM, more preferably from 2 to 10 mM and will ideally be about 5 mM.

Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, where most preferred is pH 7.6 at 25° C. Other agents which may be present in the buffer medium include chelating agents, such as EDTA, EGTA and the like.

D. Sample Housing.

The support is optionally housed in a flow chamber having an inlet and outlet to allow for renewal of reactants which flow past the immobilized moieties. The flow chamber can be made of plastic or glass and should either be open or transparent in the plane viewed by the microscope or optical reader. Electro-osmotic flow requires a fixed charge on the solid support and a voltage gradient (current) passing between two electrodes placed at opposing ends of the solid support. The flow chamber can be divided into multiple channels for separate sequencing. Examples of micro flow chambers exist. For example, Fu et al. (Nat. Biotechnol. (1999) 17:1109) describe a microfabricated fluorescence-activated cell sorter with 3 μm×4 μm channels that utilizes electro-osmotic flow for sorting.

E. Detection of Fluorophores.

Various detectors are suitable for use in the present invention. These include, but are not limited to, an optical reader, a high-efficiency photon detection system, a photodiode, a camera, a charge couple device, an intensified charge couple device, a near-field scanning microscope, a far-field confocal microscope, a microscope that detects wide-field epi-illumination, evanescent wave excitation and a total internal reflection fluorescence microscope. In certain aspects, the detection requires the imaging of single molecules in a solution. There are a variety of known ways of achieving this goal, including those described in: Basche et al., eds., 1996, “Single molecule optical detection, imaging, and spectroscopy,” Weinheim et al, “Single-molecule spectroscopy,” Ann. Rev. Phys. Chem. 48: 181-212;. Soper et al., “Detection and Identification of Single Molecules in Solution,” J. Opt. Soc. Am. B, 9(10): 1761-1769, October 1992; Keller et al. (1996), Appl. Spectrosc. 50: A12-A32; Goodwin et al. (1996), Accounts Chem. Res. 29: 607-613; Rigler (1995). J. Biotech., 41: 177; Rigler et al. Fluorescence Spectroscopy; Wolfbeis O. S., Ed.; Springer, Berlin, 1992, pp 13-24; Edman et al. (1996) Proc. Natl. Acad. Sci. USA 93: 6710; Schmidt et al. (1996) Proc. Natl. Acad. Sci. USA 1 93: 2926; Keller et al. (1996) Appl. Spectroscopy 50: A12.

A laser source is often used as the excitation source for ultrasensitive measurements but conventional light sources such as rare gas discharge lamps and light emitting diodes (LEDs) are also used. The fluorescence emission can be detected by a photomultiplier tube, photodiode or other light sensor. An array detector such as a charge-coupled device (CCD) detector can be used to image an analyte spatial distribution.

Raman spectroscopy can be used as a detection method for microchip devices with the advantage of gaining molecular vibrational information. Sensitivity has been increased through surface enhanced Raman spectroscopy (SERS) effects but only at the research level. Electrical or electrochemical detection approaches are also of particular interest for implementation on microchip devices due to the ease of integration onto a microfabricated structure and the potentially high sensitivity that can be attained. The most general approach to electrical quantification is a conductometric measurement, i.e., a measurement of the conductivity of an ionic sample. The presence of an ionized analyte can correspondingly increase the conductivity of a fluid and thus allow quantification. Amperiometric measurements imply the measurement of the current through an electrode at a given electrical potential due to the reduction or oxidation of a molecule at the electrode. Some selectivity can be obtained by controlling the potential of the electrode but it is minimal. Amperiometric detection is a less general technique than conductivity because not all molecules can be reduced or oxidized within the limited potentials that can be used with common solvents. Sensitivities in the 1 nM range have been demonstrated in small volumes (10 nL). The other advantage of this technique is that the number of electrons measured (through the current) is equal to the number of molecules present. The electrodes required for either of these detection methods can be included on a microfabricated device through a photolithographic patterning and metal deposition process. Electrodes could also be used to initiate a chemiluminescence detection process, i.e., an excited state molecule is generated via an oxidation-reduction process which then transfers its energy to an analyte molecule, subsequently emitting a photon that is detected.

Acoustic measurements can also be used for quantification of materials but have not been widely used to date. One method that has been used primarily for gas phase detection is the attenuation or phase shift of a surface acoustic wave (SAW). Adsorption of material to the surface of a substrate where a SAW is propagating affects the propagation characteristics and allows a concentration determination. Selective sorbents on the surface of the SAW device are often used. Similar techniques may be useful in the methods described herein.

In certain embodiments, the methods of the present invention involve detection of laser activated fluorescence using microscope equipped with a camera. It is sometimes referred to as a high-efficiency photon detection system. Nie et. al. (1994), “Probing individual molecules with confocal fluorescence microscopy,” Science 266:1018-1019.

The detection of single molecules involves limiting the detection to a field of view in which one has a statistical reason to believe there is only one molecule (homogeneous assays) or to a field of view in which there is only one actual point of attachment (heterogeneous assays). The single-molecule fluorescence detection of the present invention can be practiced using optical setups including near-field scanning microscopy, far-field confocal microscopy, wide-field epi-illumination, and total internal reflection fluorescence (TIRF) microscopy. For two-dimensional imaging fluorescence detection, the microscope is typically a total internal reflectance microscope. Vale et. al., 1996, Direct observation of single kinesin molecules moving along microtubules, Nature 380: 451, Xu and Yeung 1997, Direct Measurement of Single-Molecule Diffusion and Photodecomposition in Free Solution, Science 275: 1106-1109.

Suitable radiation detectors include may be, for example, an optical reader, photodiode, an intensified CCD camera, or a dye-impregnated polymeric coating on optical fiber sensor. In a preferred embodiment, an intensified charge couple device (ICCD) camera is used. The use of a ICCD camera to image individual fluorescent dye molecules in a fluid near the surface of the glass slide is advantageous for several reasons. With an ICCD optical setup, it is possible to acquire a sequence of images (movies) of fluorophores. In certain aspects, each of the NTPs of the present invention has a unique fluorophore associated with it, as such, a four-color instrument can be used having four cameras and four excitation lasers. Thus, it is possible to use this optical setup to sequence DNA. In addition, many different DNA molecules spread on a microscope slide can be imaged and sequenced simultaneously. Moreover, with the use of image analysis algorithms, it is possible to track the path of single dyes and distinguish them from fixed background fluorescence and from “accidentally dequenched” dyes moving into the field of view from an origin upstream.

In certain aspects, the preferred geometry for ICCD detection of single-molecules is total internal reflectance fluorescence (TIRF) microscopy. In TIRF, a laser beam totally reflects at a glass-water interface. The optical field does not end abruptly at the reflective interface, but its intensity falls off exponentially with distance. The thin “evanescent” optical field at the interface provides low background and enables the detection of single molecules with signal-to-noise ratios of 12:1 at visible wavelengths (see, M. Tokunaga et al., Biochem. and Biophys. Res. Comm. 235, 47 (1997) and P. Ambrose, Cytometry, 36, 244 (1999)).

The penetration of the field beyond the glass depends on the wavelength and the laser beam angle of incidence. Deeper penetrance is obtained for longer wavelengths and for smaller angles to the surface normal within the limit of a critical angle. In typical assays, fluorophores are detected within about 200 nm from the surface which corresponds to the contour length of about 600 base pairs of DNA. Preferably, a prism-type TIRF geometry for single-molecule imaging as described by Xu and Yeung is used (see, X-H.N. Xu et al., Science, 281, 1650 (1998)).

Single molecule detection can be achieved using flow cytometry where flowing samples are passed through a focused laser with a spatial filter used to define a small volume. U.S. Pat. No. 4,979,824 describes a device for this purpose. U.S. Pat. No. 4,793,705 describes and claims in detail a detection system for identifying individual molecules in a flow train of the particles in a flow cell. The '705 patent further describes methods of arranging a plurality of lasers, filters and detectors for detecting different fluorescent nucleic acid base-specific labels. U.S. Pat. No. 4,962,037 also describes a method for detecting an ordered train of labeled nucleotides for obtaining DNA and RNA sequences using a nuclease to cleave the bases rather than a polymerase to synthesize as described herein. Single molecule detection on solid supports is described in Ishikawa, et al. (1994) Single-molecule detection by laser-induced fluorescence technique with a position-sensitive photon-counting apparatus, Jan. J. Apple. Phys. 33:1571-1576. Ishikawa describes a typical apparatus involving a photon-counting camera system attached to a fluorescence microscope. Lee et al. (1994), Laser-induced fluorescence detection of a single molecule in a capillary, Anal. Chem., 66:4142-4149 describes an apparatus for detecting single molecules in a quartz capillary tube. The selection of lasers is dependent on the label and the quality of light required. Diode, helium neon, argon ion, argon-krypton mixed ion, and Nd:YAG lasers are useful in this invention.

Detecting the fluorophore can be carried out using a variety of mechanisms. These mechanisms include for example, fluorescence resonance energy transfer, an electron transfer mechanism, an excited-state lifetime mechanism and a ground-state complex quenching mechanism.

F. Labeled NTP Residence Times.

The residence time of a correctly paired NTP (i.e., an NTP that is complementary to the first unpaired nucleotide residue of the target NA that is just downstream from the extending end of the primer NA) is significantly longer than the residence time of an incorrectly paired NTP.

The kinetic mechanism has been well characterized for the reaction catalyzed by the T7 DNA polymerase. Patel et al. (1991), Biochemistry 30:511; Wong et al., Biochemistry 30:526. In this reaction, the polymerase/target NA/primer NA complex is first contacted by an NTP. When a “correct” NTP (i. e., complementary to the template nucleotide in the enzyme active site) binds, the enzyme pocket “closes” on the nucleotide and then the coupling chemistry occurs. The enzyme “opens” back up, releases the PPi formerly attached to the NTP, and the enzyme translocates to the next base on the template. An incorrect NTP (i.e., not complementary to the template base) has a very short residence time on the enzyme. See, e.g., kinetic data at Table II of Patel et al. (1991), Biochemistry 30:511. In this instance and under the polymerization conditions used, the difference between an incorporated NTP residence time is about 100 times longer to about 10,000 times longer than the residence time of an NTP that is not incorporated. In certain aspects, the residence time of an NTP that is incorporated into the primer nucleic acid is at least about 200 times longer to about 500 times longer such as 250, 350 or 450 times longer than the residence time of an NTP that is not incorporated.

The relatively long residence time of a correct NTP is used in the present invention to detect the interaction of a correct NTP with an immobilized polymerase/primer NA/template NA complex. Depending on the incubation conditions (e.g., salt concentration, temperature, pH, etc.), the residence time of a nucleotide that is incorporated into an elongating primer is longer than the residence time of an NTP that is not incorporated. The residence time of the label of a correct labeled NTP that is incorporated into the elongating primer ranges from about 1.0 milliseconds to about 100 milliseconds, preferably, from about 2.0 milliseconds to about 10 milliseconds. In certain instances, the accuracy of the residence time of the measurement depends on the speed of the detector.

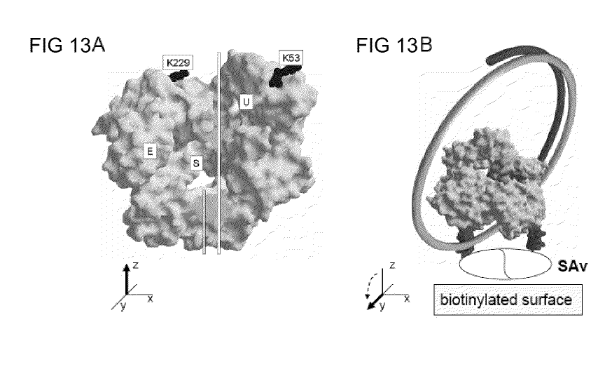

In certain preferred embodiments, the present invention provides a polymerase-DNA complex immobilized on a solid surface to enhance processivity. In a specific aspect, this was accomplished by engineering the polymerase with two biotinylated AviTag peptide legs located on either side of the DNA binding cleft. Both insertions are compatible with the protein structure of the polymerase, allowing it to be overexpressed and be biotinylated in E. coli. Stable ternary complexes of polymerase, primed template DNA and streptavidin can be immobilized on a biotinylated, non-stick surface. The expected architecture of immobilized complexes indicate that the template DNA threads through a tunnel formed by the body of the polymerase, the AviTag legs and the surface (see, FIG. 13B).

In operation, the ternary complexes (PNAC) are first assembled in solution, then purified from excess streptavidin, and finally immobilized on the surface. In an alternative embodiment, an assembly of binary complexes (biotinylated polymerase and DNA directly attached) to a surface pre-coated with streptavidin is used.

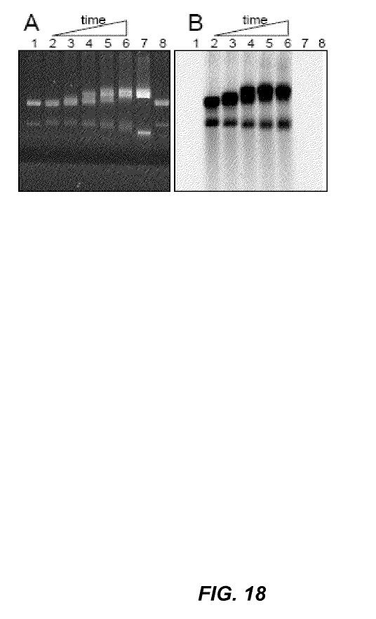

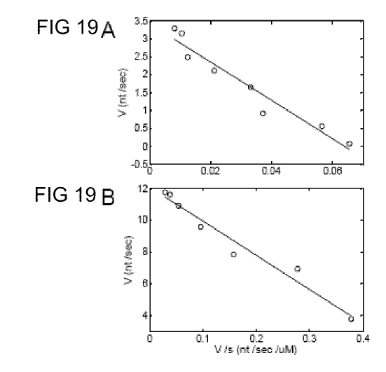

Experiments with ternary complexes in solution provided kinetic constants and processivity. In one aspect, the polymerase showed an increased requirement for dNTPs and a slower catalytic rate (2.6-fold higher Km and 3.5-fold lower Vₘₐₓ) compared to the free polymerase (see, FIG. 19). In solution, processivity was apparently enhanced from the few nucleotides characteristic of the Therminator parent polymerase, to >7 kb in the ternary complexes (see, FIG. 18). Processivity was further demonstrated by observing the activity of individual surface-attached complexes. Immobilized complexes were exposed to a cocktail of unlabeled dATP, dCTP and dGTP plus base-labeled dUTP.

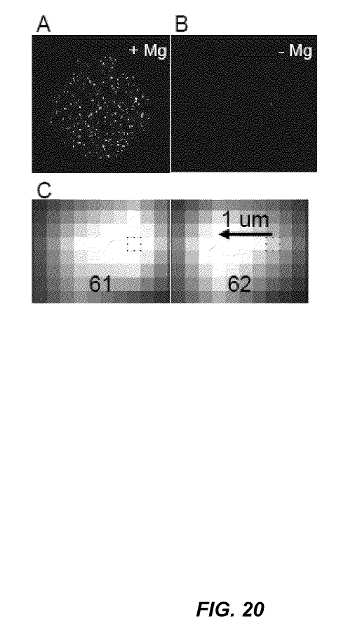

After incubation for 90 minutes, the surface is then rinsed to remove unincorporated nucleotides. DNA released from dissociated complexes is also removed, so that the only remaining DNA is that associated with immobilized complexes (FIG. 20). Individual labeled DNA spots can be seen moving back and forth even as they remained tethered to the surface. The extent of this DNA motion allows for a rough estimate of processivity. For example, FIG. 20C shows an example wherein a DNA spot appears to have moved about 1 micron between consecutive movie frames. Assuming the polymerase attachment point was at the center of this particular motion, the DNA tether fully stretched would be a minimum of 0.5 microns in length, which corresponds to 1.5 kb of DNA. Such a stretched tether, plus the apparently larger amount of DNA in the bulk fluorescent spot, indicates for a processivity of several thousand nucleotides achieved by this engineered, immobilized polymerase.

### IV. Examples

### Example 1

### Introduce a Unique Cysteine on the Protein Surface for Attaching a Fluorophore

A unique cysteine amino acid is placed on the surface of Therminator polymerase to attach the fluorescent probe. This is accomplished by site-directed mutation of the Therminator gene in two steps. First, the single native surface-exposed cysteine, C223, is eliminated by mutation to serine, resulting in the mutant C223S. Mutant C223S has no surface-exposed cysteines. Next, a new cysteine is uniquely placed on the protein surface by constructing the mutant E554C. The new cysteine is located on the rim of a cleft in the protein, near the location of a quencher on a bound nucleotide. The resulting mutant is C223S:E554C.

### Example 2

### Add Histidine Patches to the Protein Surface Attaching Anchors

Two histidine patches are engineered onto the surface of the C223S:E554C Therminator protein by making the multiple mutations D50H:T55H:E189H:R196H:K229H. The resulting mutant, C223S:E554C:D50H:T55H:E189H:R196H:K229, is called “ThioHis”.

### Example 3

### Circularization of Target DNA

Randomly-sheared fragments of genomic DNA is purified from the sample organism. The DNA is treated with T4 DNA polymerase to generate blunt ends and a single “A” nucleotide is added to the 3′-ends with Taq DNA polymerase and dATP. A mixture of two double-stranded oligonucleotide adaptors is ligated to the DNA fragments with T4 DNA ligase. See, FIGS. 3-5.

First adaptor:
Biotin-CGCCACATTACACTTCCTAACACGT
GCGGTGTAATGTGAAGGATTGTGC
Second adaptor:
CAGTAGGTAGTCAAGGCTAGAGTCT
GTCATCCATCAGTTCCGATCTCAG
Ligated DNA products:
genomic DNA: lower case
adaptors: upper case, (p) 5′-phosphate
italicized: DNA strand recovered after elution at alkaline pH
Product 1
Bio-CGCCACATTACACTTCCTAACACGTnnnnn...nnnnnaGACTCTAGCCTTGACTACCTACTGAAA-3′
GCGGTGTAATGTGAAGGATTGTGCannnnn...nnnnnTCTGAGATCGGAACTGATGGATGACp-5′
Product 2
Bio-CGCCACATTACACTTCCTAACACGTnnnnn...nnnnnaCGTGTTAGGAAGTGTAATGTGGCG-3′
3′-GCGGTGTAATGTGAAGGATTGTGCannnnn...nnnnnTGCACAATCCTTCACATTACACCGC-Bio
Product 3
5′-pCAGTAGGTAGTCAAGGCTAGAGTCTnnnnn...nnnnnaGACTCTAGCCTTGACTACCTACTGAAA-3′
3′-AAAGTCATCCATCAGTTCCGATCTCAGannnnn...nnnnnTCTGAGATCGGAACTGATGGATGACp-5′

After ligation, DNA fragments in the size range of about 17-23 kb are purified by gel electrophoresis. The purified fragments are bound to streptavidin-coated magnetic beads (Dynal). After binding, the beads are washed to remove unbound DNA. Then the bound DNA is denatured at alkaline pH and the unbiotinlyated strands are eluted (see above; Product 1, italicized font), and the DNA still bound to the beads is discarded. The eluted strands are circularized by hybridization to a primer oligo complementary to both adaptors:

| | |
|---|---|
| Primed circular template | |
| stars mark the ligation site: ** | |
| 5′-...nnnnnCGTGTTAGGAAGTGTAATGTGGCGCAGTAGGTAGTCAAGGCTAGAGTCTnnnnn...-3′ | (template strand) |
| 3′-GCACAATCCTTCACATTACACCGCGTCATCCATCAGTTCCGATCTCAGA-5′ | (primer) |

### Example 4

### Protein Modifications

The ThioHis Therminator mutant protein (Example 2) is conjugated to tetramethylrhodamine-5-maleimide (Molecular Probes) at position C554. Anchors (biotin-X nitrilotriacetic acid, Molecular Probes) are added to bind to the two histidine patches and the modified protein is purified.

### Example 5

### Anchor Protein-DNA Complexes to Glass Coverslips

The modified ThioHis protein (Example 4) is mixed with the primed circular template DNA (Example 3) to form polymerase-DNA complexes. The complexes are added to a streptavidin-coated glass coverslip to topologically trap the DNA between the protein and the glass surface. The coverslip is washed prior to sequencing the immobilized DNA.

### Example 6

### Synthesis of dUTP-γ-TMR

A. Synthesis of dUTP-γS

dUDP (16 mg, 40 μmol; Sigma D-3626) and ATP-3S (44 mg, 80 μmol; Boehringer Mannheim 102342) were dissolved in 10 mL of (20 mM Tris-Cl pH 7.0, 5% glycerol, 5 mM dithiothreitol, 5 mM MgCl₂). Nucleoside diphosphate kinase (0.5 mL, 5000 units; Sigma N-0379) was added and the sample was incubated at 37° C. for 2 h to equilibrate the γ-thiophosphate moiety between the uridine and adenosine nucleotides. As expected from the reactant stoichiometry, ⅔ of the dUDP was converted to dUTP-γS. The product was purified by reversed-phase HPLC using a linear gradient of 0% to 100% Buffer B mixed into Buffer A (Buffer A is 0.1 M triethylammonium acetate in water, pH 7, 4% acetonitrile; Buffer B is the same as Buffer A with 80% acetonitrile).

B. Synthesis of dUTP-γ-TMR

dUTP-γS (45 μg, 90 nmol; from step a) was dissolved in 295.5 μL of (20 mM sodium phosphate pH 7.5, 33% dimethylformamide). BODIPY TMRIA (4.5 μL, 0.45 μmol dissolved in dimethylformamide; Molecular Probes) was added and the sample was held in the dark at room temperature for 2.5 h. The product was obtained in 90% yield and was purified by reversed-phase HPLC as in step a.

### Example 7

### Strep-Tag II T7 DNA Polymerase

The T7 DNA polymerase gene was amplified from T7 phage DNA using the forward primer

5′-ATGATCGTTTCTGCCATCGCAGCTAAC

(encodes the exonuclease mutations A14-to-C14 and A20-to-C20) and the reverse primer

5′-TCAGTGGCAAATCGCC.

An oligonucleotide encoding the Strep-Tag II sequence overlapping the 5′-end of the amplified T7 exo- polymerase gene was synthesized on an automated oligonucleotide synthesizer:

```
5′-ATGTCCAACTGGTCCCACCCGCAGTTCGAAAAAGGTGGAGGTTCCGCT
      M S N W S H P Q F E K G G G S A
              Strep-Tag II Peptide Spacer
ATGATCGTTTCTGCCATCGCAGCTAAC..
  M I V S A I A A N....
T7 polymerase N-terminus overlap (2 exo-mutations underlined)
```

The single-stranded synthetic oligonucleotide was spliced to the amplified T7 gene (above) by overlapping PCR (Horton et al (1989) “Site-directed mutagenesis by overlap extension using the polymerase chain reaction,” Gene 77:61-68) using the StrepTag forward primer

5′-ATGTCCAACTGGTCCCACCC

with the reverse primer

5′-TCAGTGGCAAATCGCC.

The spliced PCR product was cloned into the pET11 plasmid vector (Stratagene), overexpressed in E. coli BL21(DE3)pLysS, and purified by Strep-Tag II affinity chromatography (Maier et al (1998) Anal Biochem 259: 68-73).

### Example 8

### Polymerase Immobilization

A. Surface Passivation With Polyethylene Glycol

Fused silica coverslips (1″ square, 200 μm thick; SPI Supplies, West Chester Pa.) were cleaned by soaking overnight in chromic acid and washing in distilled water in a sonic bath (Model 2200, Branson, Danbury Conn.). Methoxy-PEG-silane MW 5,000 (Shearwater Polymers, Huntsville Ala.) was dissolved at 10 mg/ml in 95:5 ethanol:water and the pH was adjusted to 2.0 with HCl. Cleaned coverslips were immersed in the PEG solution for 2 hours, washed 3 times each in ethanol, 3 times in water, dried overnight at 70 C, washed overnight in 1% sodium dodecyl sulfate in water, washed with deionized water in an ultrasonic bath, and baked for 1 day at 70 C (Jo S, Park K. Surface modification using silanated poly(ethyleneglycol)s. Biomaterials 21: 605-616. 2000).

B. Biotinylation and Streptavidin Monolayer

Photoactivatable biotin (12 μg; Pierce, Rockford Ill.) was dissolved in 1 ml of deionized water. The solution was applied to the top surface of a PEG-silane coated coverslip from step (a) and the water was evaporated under vacuum. The coverslip was exposed to UV light (General Electric Sunlamp RSM, 275W) for 20 minutes at a distance of 5 cm. The coverslip was washed with deionized water and nonspecific binding sites are blocked by overlaying a solution of 3% bovine serum albumin in 50 mM Tris-Cl pH 7.5, 150 mM NaCl (TBS) for 1 hour at room temperature. The coverslip was washed with TBS, a solution of streptavidin (1 mg/mL in TBS; Pierce, Rockford Ill.) was applied for 30 minutes, and the coverslip was washed with TBS+0.1% Tween 20 followed by TBS alone.

The streptavidin-coated coverslip from step (b) was spotted with 20 μL of T7 DNA polymerase exo⁻Strep-tag II (10 μM in TBS). After 1 hr, the coverslip was washed with TBS, ready for use.

C. Nickel Nanodots

In one embodiment, a polymerase is attached to each dot of an array of nickel nanodots. (Depending on the fluorophore used, the nickel nanodot may, however, exhibit background fluorescence, which must be corrected for.) The required equipment includes a spinner (PWM 202 E-beam resist spinner, Headway Research Inc.), an evaporator (SC4500 thermal e-gun evaporator, CVC Products Inc.), and a scanning electron microscope (Leo 982 with Nabity pattern generator, Leo Electron Microscopy Inc.).

Clean a 25 mm diameter microscope coverslip on the spinner by spraying alternately with acetone and isopropyl alcohol (IPA) and spinning the last IPA film until dry. Coat the coverslip in the spinner with 0.5 ml of PMMA (poly(methyl methylacrylate), MW 496 kDa, 2% in chlorobenzene), bake on a hotplate at 170 C for 10 min, coat with 0.5 ml of PMMA (MW 950 kDa, 2% in methyl isobutyl ketone [MIBK]), and bake again. Apply the conductive layer by evaporating 100 Angstroms of gold onto the PMMA film in the CVC SC4500. Use the electron microscope to etch the array pattern into the PMMA film using a pattern generator on the Leo 982 as specified by a CAD drawing (Design CAD, 50 nm spots, 10 μm center-to-center spacing, 200×200 dot array).

Remove the gold layer by placing the exposed coverslip in Gold Etch (15-17% sodium iodide) for 7 seconds followed by rinsing with IPA and water. Deposit Tantalum (50 Angstroms) and Nickel (100 Angstroms) on the coverslip in the CVC SC4500. Remove the PMMA in a 1:1 mix of acetone and methylene chloride for 10-15 min followed by sonication for several seconds and rinsing with IPA and water.

Attach the polymerase just before use by applying 10 μl of a 15 nM solution of polyhistidine-tagged Klenow DNA polymerase exo⁻ (prepared using TOPO cloning vector and ProBond Resin, Invitrogen Inc.) in phosphate-buffered saline (PBS; Harlow E., Lane D. 1988. Antibodies A Laboratory Manual. Cold Spring Harbor Laboratory ISBN 0-87969-14-2) to the coverslip; after 20 min, wash the coverslip in PBS and use immediately.

### Example 9

### Determination of Cystic Fibrosis Mutant

A polymerase-coated coverslip is placed on the microscope and a 20 μl sample is applied under a water immersion objective lens. The sample contains 40 mM Tris-Cl (pH 7.5), 1 mM ethylenediaminetetraacetic acid, 1 mM dithiothreitol, 0.1 mg/ml of bovine serum albumin, 12.5 mM magnesium chloride, 10 nM dUTP-TMR, 100 nM each of dATP, dCTP, and dGTP, and 10 μg/ml of primer-template DNA. Depending on the activity of the immobilized enzymes, the nucleotide concentration may have to be adjusted so that individual incorporation events are time-resolvable. Data are collected and analyzed as described in Example 6 to determine whether the dUTP-TMR nucleotide is incorporated into the primer strand. (In order to perform this experiment in a droplet on an open coverslip as described, it may be necessary to speed the motion of free dUTP-TMR through the imaged zone by drive convection with a nitrogen stream, depending on ambient conditions. It is also necessary to use a water immersion objective lens immersed directly in the sample.) The results are compared against a control without primer-template DNA to demonstrate the appearance of longer fluorescence bursts in the test sample indicating a template sequence which supports dUTP incorporation. Two sample primer-templates are compared; they are synthetic oligonucleotides derived from the cystic fibrosis transmembrane conductance regulator gene (Welsh et al. (1993), J. Cell Science 106S:235-239).

| Normal Allele (does not incorporate dUTP-γ-TMR) | |
|---|---|
| primer | 3′-CACCATTAAAGAAAATATCAT |
| template | 5′-GUGGUAAUUUCUUUUAUAGUAG |

### (Delta)F508 Deletion Mutant (Does Incorporate DUTP-γ-TMR)

| | |
|---|---|
| primer | 3′-CACCATTAAAGAAAATATCAT |
| template | 5′-GUGGUAAUUUCUUUUAUAGUAA |

### Example 10

### Microscope Setup

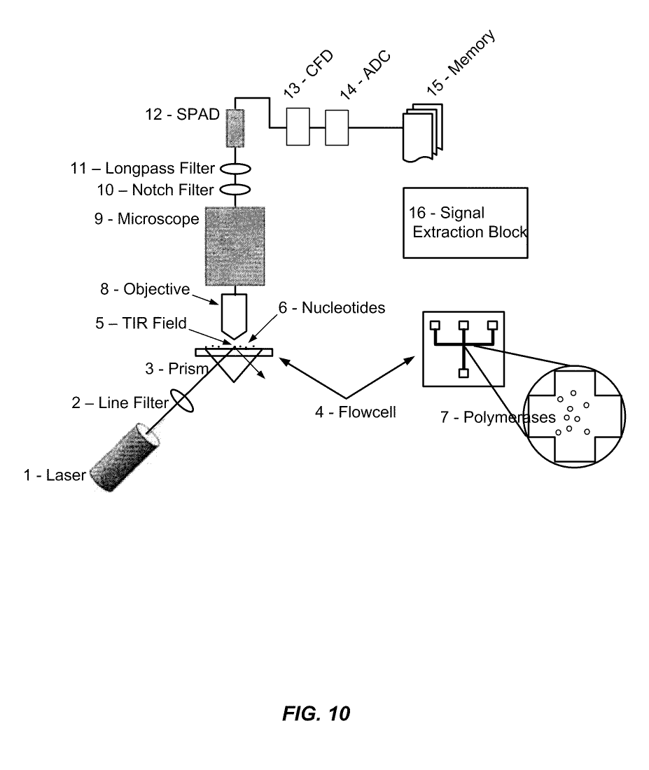

The setup for a residence-time detector is described in FIG. 10. A multicolor mixed-gas laser 1 emits light at tunable wavelengths. The laser beam is first passed through a laser line filter 2 and then at a right angle into a fused-silica prism 3 which is optically connected to the fused silica flowcell 4 by immersion oil. The labeled nucleotides 6 flow in a buffer solution across the polymerase enzymes immobilized on the surface of the flowcell chamber 7. Laser light strikes the fused silica-buffer interface at an angle such that the critical angle between fused-silica and the buffer solution is exceeded. The light is thus completely reflected at the interface, giving rise to a total internal reflection (TIR) evanescent field 5 in the solution. The angle is adjusted to give a 1/e penetrance of between 1 and 200 nm into the solution. The immobilized polymerases 7 are illuminated in the evanescent field and are imaged using a microscope 9 with an objective lens 8 mounted over the flowcell. Fluorescence emission at the microscope output passes through a notch filter 10 and a long pass filter 11 which allow the fluorescence emission to pass through while blocking scattered laser light. The fluorescence photons are focused onto a single-photon avalanche diode SPAD 12. Signals are processed by a constant fraction discriminator CFD 13, digitized by an analog-to-digital converter ADC 14, and stored in memory 15. Signal extraction algorithms 16 are performed on the data stored in memory. These algorithms may distinguish signal from background, filter the data, and perform other signal processing functions. The signal processing may be performed off-line in a computer, or in specialized digital signal processing (DSP) chips controlled by a microprocessor. The fluorescence is recorded using, for example by using CCD camera capable of recording single fluorophore molecules. Residence times and polymerase speed may be manipulated by controlling the reaction conditions (temperature, pH, salt concentration, labeled NTP concentration, etc.)

### Example 11

### Data Acquisition and Analysis

A computer model was developed to show the appearance of known (i.e., simulated) incorporation events where the nucleotide is retained by a polymerase while the base-addition chemistry occurs.

The simulation was written in MATLAB. It operates by introducing free background nucleotides into the field of view at a rate determined by the flux, which is calculated from the bias flow and optical detection volume. The detection volume is determined by the diffraction-limited focus (Airy disc diameter) and depth of the evanescent light field. The time between molecule arrivals is governed by an exponential probability distribution. As each molecule enters the simulation, the number of photons it emits is a Poisson random number, with mean calculated from the time it spends in the focal volume (determined by the bias flow), the excitation rate of the molecule (determined by the laser intensity, photon energy, and absorption cross section of the dye), and the fluorescence quantum yield of the dye. The number of photons seen by the detector is calculated in turn by the detection efficiency ratio. The photons detected are scattered in time according to a second exponential distribution, with rate calculated from the photon capture rate.

Signal molecules (i.e., nucleotides bound to the enzyme during the base-addition reaction) are introduced in time at a rate given by another simulation parameter, the reaction rate, and again distributed by a separate exponential distribution. The time a signal molecule spends in the resolution volume is determined by a random number with uniform distribution from 2 to 5 ms, consistent with the enzyme kinetics of T7 DNA polymerase (Patel S, Wong I, Johnson K (1991) Biochemistry 30: 511). The number of photons detected is a Poisson random number with mean detected as in the background molecule case. The photons detected are distributed according to the same distribution as the photons coming from background molecules.

To detect the residence-time bursts, the time arrival of all photons is discretized by a sample clock. Then the photon data is processed with a weighted sliding-sum filter, using a Hamming window. The signal energy is calculated and displayed in time. The bursts are detected by two thresholds: a signal energy threshold (vertical), and a time threshold (horizontal). A photon burst must pass both thresholds in order to be classified as a signal event.

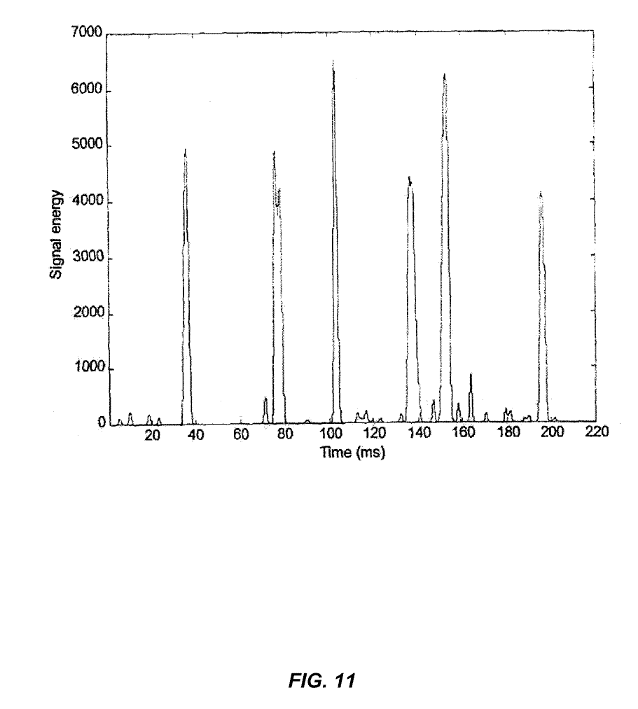

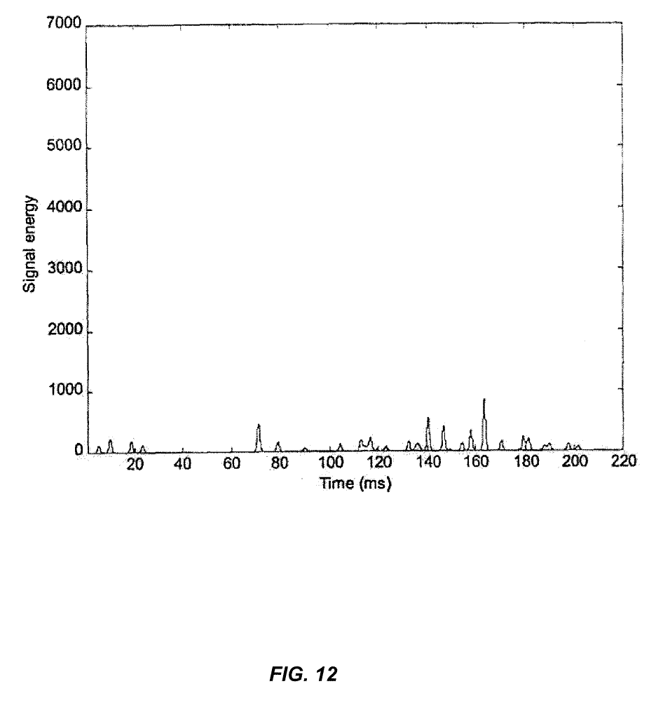

Two simulation results are shown in FIGS. 11 and 12. The parameters are the same between the two Figures (Table III).

TABLE III

| PARAMETER NAME | VALUE |
|---|---|
| Laser power | 150 (mW) |
| Laser spot diameter | 20 (micrometers) |
| Numerical aperture of objective lens | 1.2 |
| Evanescent light field height | 30 (nm) |
| Bias flow | 2 (mm/s) |
| Molarity | 10e−9 (mol/L) |
| Fluorescence quantum yield (for | 0.15 |
| Tetramethylrhodamine, TMR) | |
| Net detection efficiency | 3% |
| Sample clock | 1.0 (MHz) |

As is shown in FIG. 11, six incorporation events have occurred, all of the incorporation events are detected above a signal energy threshold of 2500. FIG. 12 corresponds to photon data from background molecules only. FIGS. 11 and 12 clearly illustrate that incorporation events and the identity of incorporated NTPs can be detected by measuring NTP residence times.

### Example 12

### Materials and Methods

### Materials

Buffer C was used for protein dilutions and polymerase assays: 10 mM Tris-Cl pH 8.0, 50 mM KCl, 0.1% Tween-20, 0.1 mM EDTA; “10×” buffer C is 10-fold concentrated Buffer C. Isoelectric focusing gels were from Invitrogen (Novex pH 3-10, Cat No. EC6655A5). Alexa Fluor-680-labeled streptavidin was from Invitrogen and unlabeled NeutrAvidin was from Pierce. Biotin-coated magnetic beads used in purifying the complexes were from Bangs Laboratories (BioMag beads cat #BM552, 1.5 μm diameter, concentration 5.2 mg/mL, binding capacity 3.5 mg streptavidin/mL).

Polymerase AviTag constructs. The starting enzyme was a mutant of Therminator DNA polymerase (http://www.neb.com) adapted by directed evolution for efficient utilization of phosphate-labeled nucleotides. AviTag is a peptide substrate for E. coli biotin-protein ligase which, when fused to a target protein, provides a site for efficient enzymatic biotinylation (http://www.avidity.com). The overlapping-primer PCR method of Chiu et al. (Chiu, J. et al., Nucleic acids research, 32, el74 (2004)) was used to insert AviTag in the mutant polymerase at two positions (Therminator coordinates K53-V54 and K229-F230). The 21-amino acid insertion ssGLNDIFEAQKIEWHEgass comprises AviTag (upper case) flanked by arbitrarily-chosen amino acids (lower case); enzymatic biotinylation occurs at the epsilon-amine of the lysine (K). The starting plasmid was a 6.4-kb pBAD-HisC plasmid (Invitrogen) containing the mutant polymerase gene.

Polymerase purification. The His-tagged polymerases were expressed from a pBAD plasmid vector (Invitrogen) either in E. coli TOP-10 (for non-biotinylated polymerase; Invitrogen) or in E. coli AVB-100 (for in-vivo biotinylation; http://www.avidity.com). Briefly, 25 mL cultures were inoculated from an overnight culture and grown under ampicillin selection to an absorbance at 600nm of 0.6-0.8 at 37° C. The cells were then induced by adding arabinose (0.04% final) and grown for an additional 4 hours at 37° C. After harvesting by centrifugation, the induced cells were incubated with 2.5 mg/mL lysozyme in Lysis Buffer (50 mM NaH₂PO₄, 300 mM NaCl, 10 mM imidazole, 0.05% Tween-20) containing protease inhibitors for 20 min on ice. Following two freeze/thaw cycles, the lysed cells were sonicated to decrease viscosity, heated to 75° C. for 15 min to denature E. coli enzymes, and pre-cleared by centrifugation. The lysate was incubated for 1 hr at 4° C. with Ni-NTA-agarose (Qiagen, Valencia, Calif.) to capture the His-tagged proteins. The resin was washed with 20 mM imidazole buffer and protein eluted with buffer containing 200 mM imidazole. The elution buffer was exchanged in one of two ways, depending on whether or not the purified polymerase was to be further biotinylated in vitro. For the parent polymerase without AviTag legs, the preparation was dialyzed overnight against storage buffer (10 mM Tris-HCl pH 8.0, 100 mM KCl, 0.1 mM EDTA, 1 mM DTT). With the imidazole now removed, the protein concentration was determined by UV absorbance (extinction coefficient calculated by amino acid composition at http://expasy.org/tools/protparam.html, giving a molar extinction coefficient for the parent polymerase of 144,000 cm⁻¹ at 280 nm), IGEPAL detergent was added to 0.02% w/v, and an equal volume of glycerol was added to a final concentration of 50% for storage at −20° C. Typical final protein concentrations were 1-5 μM in polymerase. Alternatively, to further biotinylate those polymerase variants having AviTag legs, the protein eluted in imidazole buffer was washed 3 times on a Microcon YM-30 centrifugal filter (Millipore) by first centrifuging up to 1 mL of sample at 14,000×G for 12 min. The sample was washed 3 times by re-diluting the concentrated protein in 500 μL of 10 mM Tris-Cl pH 8.0 and centrifuging. Protein concentration was measured as above and the sample was biotinylated in vitro using a kit from Avidity (cat #BIRA500) at <=12 μM in polymerase. After incubating at 30° C. for 4 hr, the sample was washed 5 times in storage buffer using a YM-30 centrifugal filter. Protein concentration was measured by UV absorbance, then IGEPAL and glycerol were added as above.

Primed M13 DNA. The DNA (20 nM) and primer (30 nM) were annealed in a 400 μL volume containing 272 μL water, 40 μL of (100 mM Tris-Cl pH 7.5, 500 mM KCl), 76 μL of M13 single-stranded DNA (104 nM; New England Biolabs) and 12 μL of either an unlabeled primer (1 μM of 5′-cgcctgcaacagtgccacgctgagagcc, desalted grade, Integrated DNA Technologies Inc.) a 5′-labled primer (1 μM of IRDye™-700 5′-cacgacgttgtaaaacgacggccagtgc, LI-COR Biosciences Inc.). The samples (4 each of 100 μL aliquots) were heated in a PCR thermocycler (MJ Research) at 95° C. for 2 min, 60° C. for 10 min, ramp 0.1° C./sec to 30° C., and were stored at −20° C.

Ternary complexes. A 50 μL sample containing 14 nM of primed M13 ssDNA, 28 nM of polymerase and 56 nM of streptavidin (PNAC) was prepared in two steps by first mixing 8 μL of water, 5 μL of 10× Buffer C, 35 μL of primed M13 ssDNA and 1.4 μL of 1M polymerase; incubating at 55° C. for 2 min; adding 0.56 μL of 5 uM streptavidin and incubating at 37° C. for 15 min. Required protein dilutions were in Buffer C. Complexes were purified from excess streptavidin (total streptavidin 2.8e-12 moles) by adding 10 μL (52 ng) of BioMag beads (streptavidin binding capacity 3.1e-12 moles/52 ng; Bangs Labs) and inverting the sample on a rotating wheel at 12 rpm for 10 min, which allows time for fast binding of free streptavidin but not for the much slower binding of the stearically encumbered complexes. The beads were removed with a magnet (Promega), the 60 μL supernatant was transferred to a new tube and the bead-binding procedure was repeated as before. The purified complexes (70 μL supernatant) was stored at 4° C. for up to a week, or stored frozen at −80° C. for future use. Assuming 100% conversion of the M13 DNA into complexes, the final concentration would be 10 nM in complexes.

Polymerization kinetics of ternary complexes. Primer extension on purified complexes was quantified as a function of the concentration of unlabeled. Purified ternary complexes were prepared as described (Ternary complexes) using the 5′-labeled primed M13 template (Primed M13 DNA). Primer extension reactions were initiated by mixing 6 μL of 2× concentrated component mix (below) plus 6 μL of 10 nM complexes. Incubation was at 54° C. for 60 sec and reactions were terminated by adding 12 μL of formamide-EDTA gel loading buffer (LI-COR cat#830-04997). The final concentration of all reaction components was 20 mM Tris-HCl pH 9.2, 50 mM KCl, 5 mM MgSO₄, 0.02% IGEPAL and 1 to 400 μM each of the 4 unlabeled dNTPs. Control samples with uncomplexed polymerase were as above, but substituting complexes for 11 nM of M13 DNA, 10 nM of labeled primer and a saturating amount of polymerase (as shown by doubling the polymerase amount with no effect on the kinetics measurement). Primer extension products were resolved by electrophoresis in a 10% polyacrylamide TBE-Urea slab gel and were detected by fluorescence (LI-COR 4200 DNA Analyzer, Lincoln, Nebr.). A size standard was included on the gel to calibrate the lengths of the primer extension products. The number-average of nucleotides added per second per complex was determined by image analysis using Image J, where the intensity of each band was weighted by its molecular size (nt); in this approach it is not necessary to know the concentration of the complexes. Plots of v vs v/S were used to determine Kₘ (-slope) and Vₘₐₓ (y-intercept), where v is nt/s and S is the molar concentration of each dNTP.

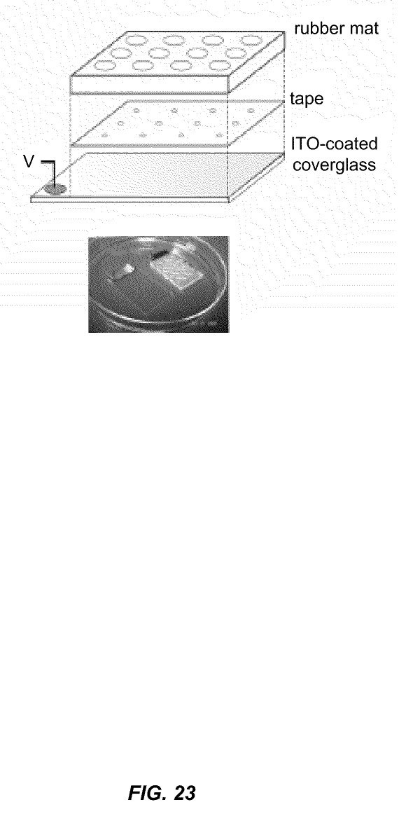

Immobilization. Microscope coverglass (Coming No. 1-1/2) was coated with indium-tin oxide (ITO, 140 ohm/square, ZC&R Inc.) film as a binder for a polyethylene oxide (PEG) non-stick coating (below). Chambers were formed on the coverglass by first applying an adhesive Mylar™ tape (70 um thick, 3×4 array of 1/32″ diameter holes, Grace Bio Labs) to the ITO, then applying an adhesive silicone rubber mat ( 1/16″ thick, ⅛″ diameter holes, Grace Bio Labs) in array-register to the tape. The ITO surface in each ⅛″ diameter chamber was coated with the polyethylene-oxide-DOPA₃ block copolymers mPEG-2000-DP3 and biotin-mPEG-3400-DP3 from Nerites Corporation (http://www.nerites.com) as follows. Each compound was dissolved under a nitrogen atmosphere at 1 mg/mL in 0.6 M K₂SO₄, 0.1 M MOPS-KOH buffer, pH 6 and stored in 200 μL single-use aliquots in sealed tubes at −80° C. A 1:8 mixture of the unbiotinylated and biotinylated compounds, respectively, was applied to the chambers. The chambers were sealed with a plastic coverslip, placed in a humid sealed jar, and incubated for 20 hr in a 60° C. oven to allow the tri-DOPA moiety to bind to the metal oxide surface. The chambers were rinsed with water and were stored dry, in the dark and in the ambient atmosphere, for up to two months before use. Purified complexes, nominally 10 nM in buffer C, were applied in 1 μL volumes to the coated chambers. The chambers were sealed with a plastic coverslip, placed in a sealed humid jar, and incubated at 50° C. for 90 min to allow the complexes to bind to the biotinylated surface. The chambers were rinsed with water and stored filled with water at 4° C. for up to 12 hr before use. One embodiment of a chamber is shown in FIG. 23.

### Processivity Clamp Design

The present invention provides an irreversible, catalytically-active complex between a polymerase and a DNA template, and orients the complexes on a nonstick surface for single DNA molecule sequencing. In certain instances, a mutant of Therminator DNA polymerase already selected by directed evolution for the efficient utilization of phosphate-labeled nucleotides is used. Like other family B DNA polymerases, Therminator binds DNA in a cleft intersecting the nucleotide binding site (Fogg, M. J. et al., Nature structural biology, 9, 922-927 (2002); Rodriguez, A. C. et al., Journal of molecular biology, 299, 447-462 (2000)) (FIG. 13A). The DNA is trapped in the polymerase by bridging the DNA binding cleft with streptavidin and binding the complex to a substrate. In one aspect, this scheme requires specific biotinylation at two surface locations on the polymerase, one on each side of the cleft. The tetravalent binding capacity of streptavidin and its strong affinity for biotin (Jung, L. S. et al., Langmuir, 16, 9421-9432 (2000)) is utilized to bind the biotinylated polymerase to a biotinylated surface.

Turning now to FIG. 13, Panel A show the DNA binding face of 9°N DNA polymerase (1QHT.pdb; (Rodriguez, A. C. et al., Journal of molecular biology, 299, 447-462 (2000)), the naturally-occurring progenitor of the modified polymerase used in this example. The primer (short white line) and template DNA (long line) strands are drawn in the DNA binding cleft. A pocket in one wall of the groove, shown in archaeal family B polymerases to bind deoxyuracil in the template strand reference, is marked “U”; the existence of this pocket provides evidence that the template tracks in the indicated cleft. AviTag peptides were inserted by the C-terminal to the marked amino acids K53 and K229. The exonuclease domain (E) and nucleotide-binding active site (S) are noted. Amino acids defining the floor ofthe groove are D4, T5, D6, Y7, 18, R17, Ki18, D235, M244, D251, K253, W342, D343, R346, S347,S348, N351, W356, V389, T590, K591, K592 and K593, and for the uracil binding pocket V93, E1111, I114, P115, P116 and RI19. Panel B is an alternate view rotated 90° about the x-axis. Circular template DNA is shown passing through the tunnel formed by polymerase and surface-bound streptavidin (SAv; the divided white oval denotes ambiguity in the number of bound streptavidins, 1 or 2). The primer strand is shown hybridized to the template. Two AviTag legs are modeled at positions K53 and K229; in the model, the length of each AviTag leg is about 25 angstroms from base to tip. The highly mobile “N” and “O” helices associated with the open-closed conformational change characteristic of polymerases are contemplated. (Rodriguez, A. C. et al., Journal of molecular biology, 299, 447-462 (2000)).

The polymerase is specifically biotinylated at the two locations flanking the DNA binding cleft. This is achieved by engineering the polymerase gene with two AviTag peptide “legs” inserted at the surface-exposed amino acid positions K53 and K229 flanking the DNA-binding cleft (FIG. 13B). AviTag is an artificial peptide substrate efficiently biotinylated by E. coli biotin-protein ligase (Beckett, D. et al., Protein Sci, 8, 921-929 (1999)) (http://www.avidity.com). The two AviTag legs facilitate biotinylation but, in projecting up to 25 Å outward from the polymerase surface, also allow ample clearance for the single-stranded template DNA to feed into the DNA binding cleft. In an animated model of RB69 DNA polymerase, the structurally-homologous locations of the two AviTag insertions in Therminator polymerase appear to be relatively rigid, with little or no participation in the major conformational changes accompanying the polymerization catalytic cycle (Steitz, T. A., The EMBO journal, 25, 3458-3468 (2006)). As such, surface attachment at these points should be compatible with polymerase activity.

### Polymerase Modification

An oligonucleotide encoding the AviTag peptide was inserted in the parent polymerase gene at one or both of the targeted locations. The progenitor gene for Therminator DNA polymerase had originally been obtained from New England Biolabs and then was re-cloned into a pBAD expression vector (Invitrogen), giving it a C-terminal hexahistidine tag to enable affinity purification with Ni-NTA beads. Five rounds of directed evolution resulted in 7 new mutations adapting the polymerase to the efficient utilization of phosphate-labeled nucleotides. Starting with this polymerase, constructs having an AviTag leg inserted between K53-V54 (construct “B53”), between K229-F230 (“B229”), or at both locations (“DBio”) were prepared. Candidate constructs were screened by PCR from E. coli colonies and were confirmed by DNA sequencing. The modified proteins were expressed in E. coli AV101 in order to biotinylate the AviTag legs in vivo (http://www.avidity.com). The engineered polymerases were affinity purified using Ni-NTA beads and then further biotinylated in vitro with biotin-protein ligase (Materials and Methods). The biotinylated polymerases were evaluated for purity on an SDS-PAGE gel.

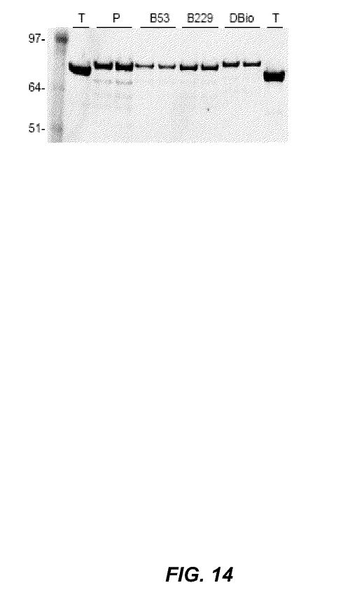

Turning now to FIG. 14, a gel showing purified polymerases with AviTag legs are shown. Lane (T) is Therminator protein commercially available from New England Biolabs. Lane (P) is the parent enzyme (a mutant of Therminator selected for improved utilization of phosphate-labeled dNTPs) which has a higher molecular weight compared to Therminator because it has a C-terminal Myc-His fusion used for purification. Lane (B53) is polymerase “P” with AviTag leg at position K53. Lane (B229) is polymerase “P” with AviTag leg at position 229. Lane (DBio; “dual biotin”) is mutant “P” with two AviTag insertions at K53 and K229. Duplicate samples (5 μL each) of the Ni-NTA purified proteins were resolved by SDS-PAGE (Invitrogen NuPAGE Bis-Tris (MOPS) 4-12%). The gel was stained with Coomassie Blue and imaged with a LI-COR Odyssey infrared imager. Marker sizes (kDa) are indicated.

### Binary Complexes of Polymerase and Streptavidin

To estimate the extent of biotinylation, increasing amounts of the DBio polymerase (0-10 nM) with a fixed amount of fluorophore-labeled streptavidin (0.5 nM) are mixed. The obtained complexes were resolved in an isoelectric focusing gel and bands containing labeled streptavidin were imaged using a LI-COR infrared fluorescence imager. The amount of bound streptavidin increased with polymerase concentration, with nearly all of the streptavidin bound by a 2-fold molar excess of polymerase. This indicates that at least half of the polymerase proteins are biotinylated and capable of binding streptavidin, which is sufficient to proceed with DNA binding experiments.

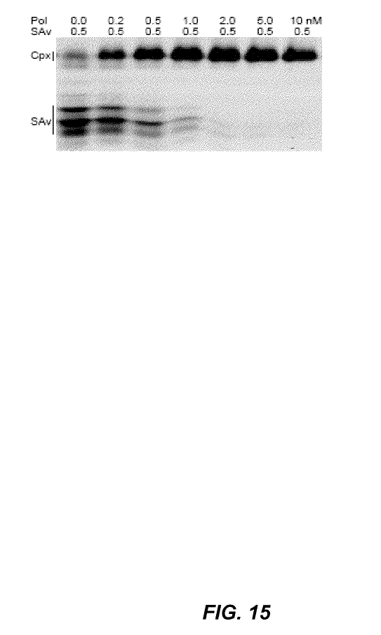

Turning now to FIG. 15, binary complexes of polymerase and streptavidin are shown. Complexes were formed by incubating 0.5 nM Alexa Fluor-680 labeled streptavidin (Invitrogen) plus 0.2-10.0 nM purified DBio polymerase (i.e., two legs) at 37° C. for 10 min. The complexes were resolved by isoelectric focusing and the gel as shown was scanned using a LI-COR Odyssey infrared imager. The positions of the binary complexes (Cpx) and unbound streptavidin (SAv) are indicated. A small fraction of the unbound streptavidin appears to have the same isoelectric point as the binary complexes in the first lane.

### Ternary Complexes of Polymerase, DNA and Streptavidin

All four polymerase variants were tested for the ability to form stable ternary complexes with DNA and streptavidin. In this experiment, primed M13 DNA, polymerase and labeled streptavidin were mixed in a molar ratio of 1:2:4, respectively. The DNA and polymerase were mixed first, then streptavidin was added with the idea of trapping the DNA in the polymerase.

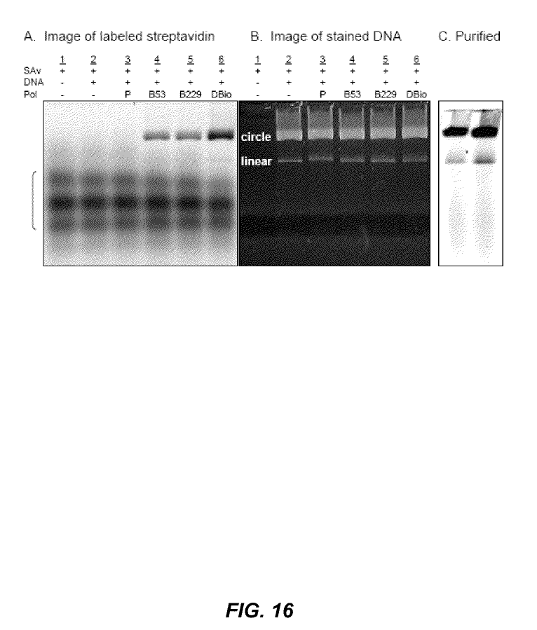

Turning now to FIG. 16, a gel of ternary complexes is shown made with primed M13 DNA, polymerase (zero, one or two AviTag legs) and Alexa Fluor-680-streptavidin in lanes 3-6, with controls omitting either DNA or polymerase in lanes 1 and 2. Complexes were separated by electrophoresis in a 2.2% agarose gel (10 v/cm, 1 hr). In panel A, the tested polymerases were the parent enzyme without AviTag legs (P); the two single-leg variants (B53 and B229); and the dual-leg polymerase (DBio). The labeled streptavidin (SAv) was detected using a LI-COR Odyssey infrared imager. The relative quantities of streptavidin (SAv) associated with both circular and linear DNA were determined by integrating pixel intensities: lane 3 (SAv=0), lane 4 (SAv=1.0), lane 5 (SAv=1.0) and lane 6 (SAv=2.1). Panel B shows a gel from (A), but stained with SYBR Gold™ (Invitrogen) to visualize the DNA by UV transillumination (312 nm). Panel C shows a gel of purified complexes of DBio polymerase (infrared image), wherein the unbound labeled streptavidin has been removed compare to the unpurified complexes in A, lane 6. The products were resolved by agarose gel electrophoresis and the infrared fluorescence signal was imaged to reveal the labeled streptavidin (FIG. 16A).

The DNA was separately imaged by staining the gel with SYBR Gold and photographing under UV illumination (FIG. 16B). Complexes were identified as labeled streptavidin co-migrating with either the circular or linear M13 DNA. Importantly, the ternary complexes are observed and their formation depends on the presence of a polymerase with at least one AviTag leg. So, although the polymerase is unlabeled and thus not directly detectable, the requirement for biotinylated polymerase in binding labeled streptavidin to DNA establishes that the complexes comprise all 3 components. Approximately twice (2.1-fold) as much streptavidin was bound by the DBio polymerase as compared to the single-leg variants (FIG. 16A), suggesting that there is one streptavidin bound per leg. That is, the protein configuration may prevent a single streptavidin spanning the two legs.

### Purification

To minimize competitive binding to biotinylated surfaces, the ternary complexes were purified from the excess of streptavidin used in preparation. A procedure is developed based on the faster binding of streptavidin to biotinylated magnetic beads as compared to the slower binding of the bulky ternary complexes. In this procedure, biotinylated magnetic beads are added to unpurified complexes in sufficient capacity to bind all of the streptavidin present. The sample is mixed for 10 min at room temperature, the beads are magnetically removed, and the process is repeated once. These purified complexes are largely free of excess streptavidin (FIG. 16C). No additional label is removed by further cycles of purification, indicating that essentially all of the functional streptavidin is removed by the two cycles of bead purification. With the complexes being formed from a 1:2:4 mixture of DNA:polymerase:streptavidin, most of the polymerase not associated with DNA is likely to be in binary complex with streptavidin.

### Thermal Stability

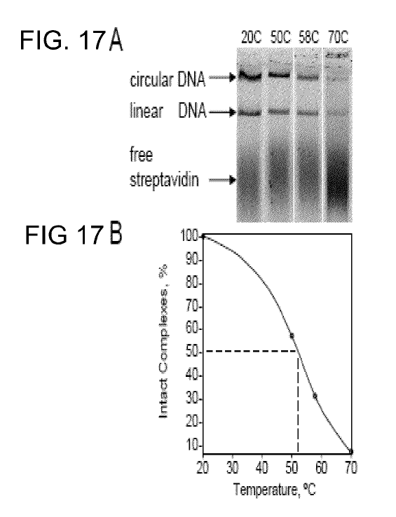

The thermal stability of ternary complexes are determined to see if they could be used near the 74° C. temperature optimum of the polymerase. Turning to FIG. 17, purified complexes (3 nM) were incubated for 2 hr at the indicated temperatures and samples were resolved by electrophoresis in 2.2% agarose. Panel A shows a gel of the labeled streptavidin component imaged using an Odyssey infrared imager. Panel B shows for each lane, the fluorescence signals co-migrating with the two DNA bands (circular, linear) summed and the results plotted normalized to the 20° C. sample. The data points were connected by a piecewise spline curve.

Purified ternary complexes made with labeled streptavidin were incubated between 20 and 70° C. for 2 hr and the fraction of complexes surviving intact was determined by agarose gel electrophoresis (FIG. 17A). Interpolating the plot in FIG. 17B, 54° C. is optimal for testing the polymerization activity of ternary complexes, where about 45% of the complexes survived intact after 2 hours incubation and where polymerase activity is about ⅓ the maximum at 74° C. The apparent stabilities measured here could have been affected by reassociation occurring in the time period (approximately 10 min) between cooling the samples and separating the components by electrophoresis.

### Activity of Ternary Complexes in Solution

Purified complexes were incubated with unlabeled dNTPs to allow for primer extension on the associated M13 templates. Samples were incubated at 54° C. for times up to 90 min and primer extension products were resolved on an agarose gel. DNA products were imaged by staining with SYBR Gold™.

Turning now to FIG. 18, gels representing DNA synthesis by ternary complexes are shown. Complexes (1.2 nM) made with labeled streptavidin were mixed with 200 μM each of the 4 unlabeled dNTPs and 5 mM MgCl₂ in buffer C and were incubated at 54° C. for 0, 3, 10, 30 and 90 min (lanes 2-6). The samples were resolved by electrophoresis in a 2.2% agarose gel. Panel A shows the DNA component stained with SYBR Gold™ and imaged under UV transillumination. Panel B shows the streptavidin component was imaged by fluorescence using a LI-COR Odyssey infrared imager. Controls include M13 DNA alone (lanes 1 and 8), and M13 fully extended with a saturating amount of Taq DNA polymerase (lane 7).

The complexes appear capable of highly-processive DNA synthesis, as indicated by the full-length products obtained at the longer incubation times (FIG. 18A, lanes 2-6). The majority of the labeled streptavidin remained associated with the DNA product bands, which suggests that the complexes remained mostly intact for the 90 min duration of the reaction (FIG. 18B). There was little or no evidence for strand displacement synthesis by the complexes, which would be revealed as high molecular weight DNA trapped in the wells as seen, for example, with the strand-displacing DNA polymerase φ-29. The ability to detect the strand-displacing nature of DNA polymerase φ-29 using this system was confirmed was confirmed with additional experiments.

### Polymerization Kinetics in Solution

Kinetic constants of purified complexes were determined in a primer extension assay. In this experiment, the streptavidin was unlabeled and an infrared dye-labeled primer was used to the detect primer extension products with single-base resolution. Samples were incubated for 60 sec at 54° C. with unlabeled dNTP concentrations of from 1 to 400 μM each. Primer extension products were resolved by electrophoresis in an automated sequencer (LI-COR 4200). Each lane on the gel was analyzed to quantify the average number of nucleotides incorporated per template molecule, and polymerization rates were calculated at each dNTP concentration. A reciprocal plot gave Km_{└dNTp┘}=54 μM and Vₘₐₓ=3.4 nt/s for the purified complexes (FIG. 19). DNA synthesis rates (v, nt/sec) were determined for various nucleotide substrate concentrations (s, μM). Panel A shows the purified complexes: Km=54 μM, Vmax=3.4 nt/s; and Panel B shows the control sample of uncomplexed polymerase and DNA: Km=21 μM, Vmax=12.0 nt/s. By comparison, a control experiment using free, uncomplexed polymerase had a greater affinity for nucleotides and a faster polymerization rate (Km_{└dNTP┘}=21 μM and Vₘₐₓ=12.0 nt/s measured at 54° C.)

### Activity of Immobilized Complexes

The thermal stability and activity of ternary complexes in solution are capable of processive DNA synthesis. It was determined that the complexes are active when immobilized on a surface. Microscope coverglass chambers were coated with a 1:8 mixture of biotinyl-PEG3400 and PEG2000 polymers to provide a nonstick surface capable of specifically binding streptavidin. A solution of unlabeled ternary complexes was applied to the chambers and allowed to bind for 90 minutes. The chambers were washed to remove unbound complexes and a nucleotide cocktail containing base-labeled AlexaFluor-488-dUTP plus unlabeled dATP, dCTP and dGTP was added. After incubation at 54° C. for 2½ hours, the chambers were washed to remove free nucleotides and the coverglass surface was imaged by TIRF microscopy. A field of bright fluorescent spots was seen (FIG. 20A). FIG. 20 shows purified ternary complexes made with unlabeled immobilized polymerase in a reaction chamber on a PEG-biotin coated coverglass. Panel A shows the reaction chamber was filled with 1 μL of buffer C containing 5 mM MgCl₂, 100 μM each of dATP, dCTP, dGTP and base-labeled Alexa Fluor-488-dUTP (Invitrogen).

The chamber was sealed with a plastic coverslip and incubated in a humid jar at 54° C. for 90 min. The chamber was rinsed with water to remove unincorporated nucleotides and the coverglass surface was imaged by TIRF microscopy. The labeled complexes were seen waving back and forth under Brownian motion while remaining tethered to the surface. Panel B shows the control reaction inhibiting polymerase activity by replacing Mg⁺⁺ with 0.1 mM EDTA. Panel C shows zoomed-in view of a single DNA spot showing movement of about 1 μm leftward occurring between frames 61 and 62. A pixel is marked for reference (x-y coordinate 386,534). Exposure time was 80 msec and the pixel dimension 0.27 microns.

Each spot marks a single, multiply-labeled DNA molecule synthesized by surface-attached complexes. This conclusion is supported by two additional observations ruling out the possibility that the bright spots are non-specifically adsorbed clusters of labeled dUTP. First, no spots were observed in a negative control omitting Mg⁺⁺, which is required for base incorporation (FIG. 20B). Secondly, the spots were seen moving freely back and forth while still remaining tethered to the surface (FIG. 20C). This behavior is consistent with individual, labeled DNA molecules retained by individual polymerases.

### Complexes Can Be Stored Frozen For Subsequent Use

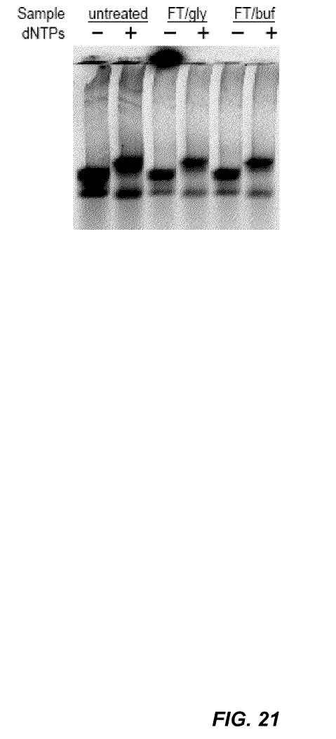

The complexes can be stored for up to 1 week at 4° C. with minimal loss in activity. For purposes of single-molecule DNA sequencing, however, it would be more convenient to prepare complexes with genomic DNA samples in advance and store them indefinitely until needed. To see if pre-formed complexes could be frozen without losing activity, we prepared samples in buffer alone or in buffer plus 25% glycerol. Aliquots were frozen in liquid nitrogen, stored overnight at −80° C., and thawed at ambient temperature. Samples were tested by incubating with unlabeled dNTPs at 54° C. for 30 minutes and analyzed on an agarose gel (FIG. 21).

As shown in FIG. 21, purified complexes (3 nM) made with Alexa Fluor-680 streptavidin were treated by freezing 20 μL aliquots in liquid nitrogen, storing overnight at −80 and thawing at 20° C. Treated samples (FT) were in buffer C alone (buf) or in buffer C plus 25% glycerol (gly). The thawed complexes were tested for activity by incubating 2 μL of complexes in the presence or absence (±) of 200 μM each of dATP, dCTP, dGTP and dTTP in a final volume of 10 μL buffer C at 55° C. for 30 min. Primer extension products were resolved by electrophoresis in a 1.5% agarose gel and the labeled streptavidin component was imaged using a LI-COR Odyssey infrared imager. An unfrozen control was held overnight at 4° C. (untreated).

Both of the frozen samples not only survived intact, but also showed full activity indistinguishable from a not-frozen control. Since damage to complexes is most likely to occur during freezing and thawing, we believe that these engineered complexes could be frozen indefinitely until needed.

### Polymerase and Expression Vector

Nucleotide sequence of the plasmid used to construct the AviTag leg insertions (6379 bp) is set forth in the informal sequence listing. The polymerase gene (upper case) is cloned in a pBAD-HisC vector (lower case; Invitrogen). For reference, the appended 6× His tag is encoded (CAT)₆ at the 3′-end of the polymerase gene. The AviTag insertion made between amino acids K53-V54 divides the dinucleotide GG at 477-478 (bold text; numbered from 1 in the sequence below), and the insertion between amino acids K229-F230 divides GT at 1005-1006.

### Polymerase AviTag™ Constructs.

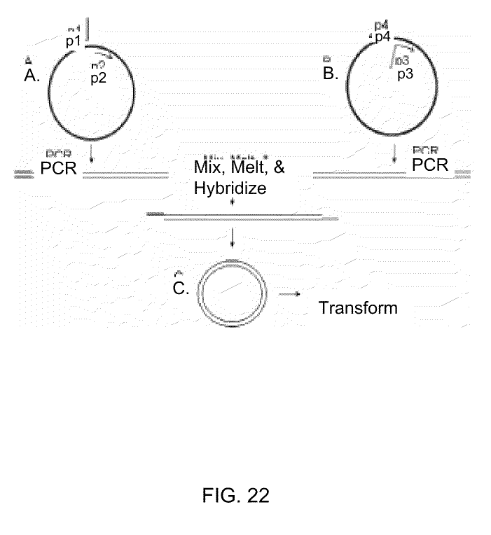

To insert an AviTag peptide at a single position in the polymerase, the entire plasmid vector was first amplified in two separate PCR reactions (FIG. 22), which were then heteroduplexed prior to transformation into E. coli. The AviTag insertion is encoded by primers p1 and p3 (below) in their 5′-tails; the nucleotide sequences of the two tails are mutually complementary.

FIG. 22 schematically illustrates the approach of Chiu et al. (Chiu, J., March, P. E., Lee, R., Tillett, D. 2004.) Site-directed, Ligase-Independent Mutagenesis (SLIM), wherein a single-tube methodology approaching 100% efficiency in 4 hours. Nucleic Acids Res 32: e174) that was used for inserting the AviTag legs.

The procedure for constructing the K53-V54 insertion follows:

Primers were from Integrated DNA Technologies Inc. and were PAGE-purified by the vendor prior to use.

p1:gctagatgcgccttcgtgccattcgattttctgagcttcgaagatgtcgttcagaccgctagacttctt
gacgtcctctatcgcag,
p2:cttcttgacgtcctctatcgcag,
p3:tctagcggtctgaacgacatcttcgaagctcagaaaatcgaatggcacgaaggcgcatctagcgt
aaccgcaaagaggcacgg,
p4:gtaaccgcaaagaggcacgg

The 25 μL PCR reactions contained plasmid DNA (8 pg/uL), 2 mM MgSO₄, 20 mM Tris-Cl pH 9.0, 50 mM KCl, 0.4 μM each of the two primers, 0.2 mM each dNTP, 80 units/mL of Taq DNA polymerase (Promega) and 20 units/mL of Pfu Ultra DNA polymerase (Stratagene). The thermal cycling schedule was 95° C. for 2 min, 10 cycles of (95 ° C. for 15 sec, 58° C. for 30 sec, 68° C. for 6 min), 20 cycles of (95° C. for 15 sec, 58° C. for 30 sec, 68° C. for 6 min extended by 5 sec each cycle), 68 ° C. for 5 min. A 1-μL volume of Dpn I (20,000 units/mL, New England Biolabs) was added and both samples were incubated at 37° C. for 60 min. The samples were resolved by electrophoresis in a 1.2% agarose gel (eGel, Invitrogen); the major product was the size of full-length plasmid DNA. The full-length product bands were purified and recovered in a 50 μL volume (Qiagen Gel Extraction Kit). The purified DNA products were mixed in equal volumes (4 μL of each in a 30 μL final volume containing 100 mM NaCl plus 20 mM Tris-Cl pH 8.0) and were heated at 99° C. for 3 min followed by 2 cycles of (65° C. for 5 min, 30° C. for 15 min). Chemically-competent E. coli TOP 10 cells (Invitrogen) were transformed with 8 μL of the annealed sample and selected for ampicillin resistance. Clones were screened by transferring single colonies to 50 μL of water and amplifying in 25 μL reaction mixtures containing 1 μL of cell suspension, 5 mM MgSO₄, 20 mM Tris-Cl pH 9.0, 50 mM KCl, 0.4 μM each of diagnostic primers 5′-ccttctgaaggacgattctgcg and 5′-cgcttcaccttgacaaccg, and Taq DNA polymerase (20 units/mL); amplification conditions were 95° C. for 2 min, 25 cycles of (95° C. for 15 sec, 54° C. for 30 sec, 68° C. for 1 min), 68° C. for 5 min. Samples were resolved by gel electrophoresis; clones containing the desired insert were identified as having a 180-bp amplicon, whereas negative clones had a 117-bp amplicon. Nine of eleven clones were positive by this PCR test. Two clones were confirmed by DNA sequencing. The first was the desired sequence while the second had an unintended mutation and was discarded.

The same procedure was used for inserting the second AviTag peptide between K229-F230, starting either from the same parent gene as above (to construct a single leg insertion at K229) or from the K53-V54 insert obtained above (to add the second leg at K229). Primers for the second insertion were:

p1:gctagatgcgccttcgtgccattcgattttctgagcttcgaagatgtcgttcagaccgctagactttattccgagttcctcacagcg, p2: ctttattccgagttcctcacagcg, p3:tctagcggtctgaacgacatcttcgaagctcagaaaatcgaatggcacgaaggcgcatctagcttcacactcggcagggacgg, p4: ttcacactcggcagggacgg, with the diagnostic primers ttcgctgtatcttcggctcg and tacctgaagaagcgctgtgag. For both the single and the double leg constructs, the same high yield of positive clones was obtained (˜90%) and individual clones were confirmed by sequencing.

All publications and patent applications cited in this specification are herein incorporated by reference as if each individual publication or patent application were specifically and individually indicated to be incorporated by reference.

aagaaaccaattgtccatattgcatcagacattgccgtcactgcgtcttt
tactgcctcttctcgctaaccaaaccggtaaccccgcttattaaaagca
ttctgtaacaaagcgggaccaaagccatgacaaaaacgcgtaacaaaag
tgtctataatcacggcagaaaagtccacattgattatttgcacggcgtc
acactttgctatgccatagcatttttatccataagattagcggatccta
cctgacgctttttatcgcaactctctactgtttctccatacccgttttt
tgggctaacaggaggaattacatATGATTCTCGATACCGACTACATCAC
CGAGAACGGGAAGCCCGTGATAAGGGTCTTCAAGAAGGAGAACGGCGAG
TTTAAAATCGAGTACGACAGAACCTTCGAGCCCTACTTCTACGCCCTTC
TGAAGGACGATTCTGCGATAGAGGACGTCAAGAAGGTAACCGCAAAGAG
GCACGGAACGGTTGTCAAGGTGAAGCGCGCCGAGAAGGTGCAGAAGAAG
TTCCTCGGCAGGCCGATAGAGGTCTGGAAGCTCTACTTCAACCATCCTC
AGGACGTCCCGGCGATTCGAGACAGGATACGCGCCCACCCCGCTGTCGT
TGACATCTACGAGTACGACATACCCTTCGCCAAGCGCTACCTCATCGAC
AAGGGCCTGATTCCGATGGAGGGCGACGAGGAGCTTACGATGCTCGCCT
TCGCGATCGCAACCCTCTATCACGAGGGCGAGGAGTTCGGAACCGGGCC
GATTCTCATGATAAGCTACGCCGACGGGAGCGAGGCGAGGGTGATAACC
TGGAAGAAGATTGACCTTCCGTACGTTGACGTCGTCTCGACCGAGAAGG
AGATGATTAAGCGCTTCCTCCGCGTCGTCAGGGAGAAGGACCCCGACGT
GCTCATCACCTACAACGGCGACAACTTCGACTTCGCCTACCTGAAGAAG
CGCTGTGAGGAACTCGGAATAAAGTTCACACTCGGCAGGGACGGGAGCG
AGCCGAAGATACAGCGAATGGGCGACCGCTTTGCCGTTGAGGTGAAGGG
CAGGATTCACTTCGACCTCTACCCCGTCATAAGGCGCACGATAAACCTC
CCGACCTACACCCTTGAGGCCGTTTACGAGGCCGTCTTTGGAAAGCCCA
AGGAGAAGGTTTACGCAGAGGAGATAGCGCAGGCCTGGGAGAGCGGGGA
GGGCCTTGAAAGGGTTGCAAGATACTCGATGGAGGACGCTAAGGTGACC
TACGAGCTGGGAAGGGAGTTCTTCCCGATGGAGGCCCAGCTTTCGAGGC
TTATAGGCCAGAGCCTCTGGGACGTCTCGCGCTCGAGCACCGGAAATTT
GGTGGAGGCATTCCTCCTGCGGAAGGCCTACAAGAGGAACGAGCTCGCC
CCAAACAAGCCCGACGAGAGGGAGCTCGCGAGACGGCGCGGGGGCTACG
CTGGCGGGTACGTTAAGGAACCAGAGCGGGGATTGTGGGACAACATTGT
GTATTTAGACTTCCGCTCGTGGTATCCTTCAATCATCATAACCCACAAC
GTCTCGCCGGATACCCTCAACCGCGAGGGCTGTAAAGAGTACGACGTCG
CCCCTGAGGTTGGACACAAGTTCTGCAAGGACTTCCCCGGCTTCATACC
AAGCCTCCTGGGAGATTTGCTCGAGGAGGCGAGCAAGATAGAGCGGAAG
ATGAAGGCAACGGTTGACCCGCTGGAGAAGAAACTCCTCGTGTACAGGC
AGTGGCTTATAAAAATCCTCGCCAACAGCTTCTACGGCTACTACGGCTA
CGCCAAGGCCCGGTGGTACTGCAAGGAGTGCGCCGAGAGCGTTACGGCC
TGGGGAAGGGAGTATATAGAAATGGTTATCCGGGAACTCGAAGAAAAAT
TCGGTTTTAAAGTTCTCTATGCCGATACAGACGGTCTCCATGCTACCAT
TCCCGGAGCAGACGCTGAAACAGTCAAGAAAAAAGCAAAGGAGTTCTTA
AAATACATTAATCCAAAACTGCCCGGCCTGCTCGAACTTGAGTACGAGG
GCTTCTACGTGAGGGGCTTCTTCGTCACGAAGAAGAAGTACGCTGTGAT
AGACGAGGAGGGCAAGATAACCACGAGGGGTCTTGAGATTGTGAGGCGC
GACTGGAGCGAGATAGCGAAGGAGACCCAGGCCAGGGTCTTAGAGGCGA
TACTCAAGCACGGTGACGTCGAGGAGGCCGTTAGGATAGTCAAGGAAGT
GACGGAAAAGCTGAGCAAGTATGAGGTCCCGCCCGAGAAGCTGGTAATC
CACGAGCAGATAACGCGCGATTTGAGGGATTACAAAGCCACCGGCCCGC
ACGTTGCCGTTGCGAAGAGGCTCGCGGCGCGTGGAGTGAAAATCCGGCC
CGGCACGGTGATAAGCTACATCGTCCTAAAGGGCTCTGGAAGGATAGGC
GACAGGGCGATTCCAGCTGATGAGTTCGACCCGACGAAGCACCGCTACG
ATGCGGAATACTACATCGAGAACCAGGTTCTCCCGGCGGTGGAGAGGAT
TCTAAAAGCCTTCGGCTATCGGAAGGAGGATTTGCGCTACCAGAAGACG
AAGCAGGTCGGCTCGGGCGCGTGGCTGAAGGTGAAGGGGAAGAAGGGTA
CCGAAGCTTACGTAGAACAAAAACTCATCTCAGAAGAGGATCTGAATAG
CGCCGTCGACCATCATCATCATCATCATTGAgtttaaacggtctccagc
ttggctgttttggcggatgagagaagattttcagcctgatacagattaa
atcagaacgcagaagcggtctgataaaacagaatttgcctggcggcagt
agcgcggtggtcccacctgaccccatgccgaactcagaagtgaaacgcc
gtagcgccgatggtagtgtggggtctccccatgcgagagtagggaactg
ccaggcatcaaataaaacgaaaggctcagtcgaaagactgggcctttcg
ttttatctgttgtttgtcggtgaacgctctcctgagtaggacaaatccg
ccgggagcggatttgaacgttgcgaagcaacggcccggagggtggcggg
caggacgcccgccataaactgccaggcatcaaattaagcagaaggccat
cctgacggatggcctttttgcgtttctacaaactcttttgtttattttt
ctaaatacattcaaatatgtatccgctcatgagacaataaccctgataa
atgcttcaataatattgaaaaaggaagagtatgagtattcaacatttcc
gtgtcgcccttattcccttttttgcggcattttgccttcctgtttttgc
tcacccagaaacgctggtgaaagtaaaagatgctgaagatcagttgggt
gcacgagtgggttacatcgaactggatctcaacagcggtaagatccttg
agagttttcgccccgaagaacgttttccaatgatgagcacttttaaagt
tctgctatgtggcgcggtattatcccgtgttgacgccgggcaagagcaa
ctcggtcgccgcatacactattctcagaatgacttggttgagtactcac
cagtcacagaaaagcatcttacggatggcatgacagtaagagaattatg
cagtgctgccataaccatgagtgataacactgcggccaacttacttctg
acaacgatcggaggaccgaaggagctaaccgcttttttgcacaacatgg
gggatcatgtaactcgccttgatcgttgggaaccggagctgaatgaagc
cataccaaacgacgagcgtgacaccacgatgcctgtagcaatggcaaca
acgttgcgcaaactattaactggcgaactacttactctagcttcccggc
aacaattaatagactggatggaggcggataaagttgcaggaccacttct
gcgctcggcccttccggctggctggtttattgctgataaatctggagcc
ggtgagcgtgggtctcgcggtatcattgcagcactggggccagatggta
agccctcccgtatcgtagttatctacacgacggggagtcaggcaactat
ggatgaacgaaatagacagatcgctgagataggtgcctcactgattaag
cattggtaactgtcagaccaagtttactcatatatactttagattgatt
taaaacttcatttttaatttaaaaggatctaggtgaagatcctttttga
taatctcatgaccaaaatcccttaacgtgagttttcgttccactgagcg
tcagaccccgtagaaaagatcaaaggatcttcttgagatcctttttttc
tgcgcgtaatctgctgcttgcaaacaaaaaaaccaccgctaccagcggt
ggtttgtttgccggatcaagagctaccaactctttttccgaaggtaact
ggcttcagcagagcgcagataccaaatactgtccttctagtgtagccgt
agttaggccaccacttcaagaactctgtagcaccgcctacatacctcgc
tctgctaatcctgttaccagtggctgctgccagtggcgataagtcgtgt
cttaccgggttggactcaagacgatagttaccggataaggcgcagcggt
cgggctgaacggggggttcgtgcacacagcccagcttggagcgaacgac
ctacaccgaactgagatacctacagcgtgagctatgagaaagcgccacg
cttcccgaagggagaaaggcggacaggtatccggtaagcggcagggtcg
gaacaggagagcgcacgagggagcttccagggggaaacgcctggtatct
ttatagtcctgtcgggtttcgccacctctgacttgagcgtcgatttttg
tgatgctcgtcaggggggcggagcctatggaaaaacgccagcaacgcgg
cctttttacggttcctggccttttgctggccttttgctcacatgttctt
tcctgcgttatcccctgattctgtggataaccgtattaccgcctttgag
tgagctgataccgctcgccgcagccgaacgaccgagcgcagcgagtcag
tgagcgaggaagcggaatagcgcctgatgcggtattttctccttacgca
tctgtgcggtatttcacaccgcatctggtgcactctcagtacaatctgc
tctgatgccgcatagttaagccagtatacactccgctatcgctacgtga
ctgggtcatggctgcgccccgacacccgccaacacccgctgacgcgccc
tgacgggcttgtctgctcccggcatccgcttacagacaagctgtgaccg
tctccgggagctgcatgtgtcagaggttttcaccgtcatcaccgaaacg
cgcgaggcagcagatcaattcgcgcgcgaaggcgaagcggcatgcataa
tgtgcctgtcaaatggacgaagcagggattctgcaaaccctatgctact
ccgtcaagccgtcaattgtctgattcgttaccaattatgacaacttgac
ggctacatcattcactttttcttcacaaccggcacggaactcgctcggg
ctggccccggtgcattttttaaatacccgcgagaaatagagttgatcgt
caaaaccaacattgcgaccgacggtggcgataggcatccgggtggtgct
caaaagcagcttcgcctggctgatacgttggtcctcgcgccagcttaag
acgctaatccctaactgctggcggaaaagatgtgacagacgcgacggcg
acaagcaaacatgctgtgcgacgctggcgatatcaaaattgctgtctgc
caggtgatcgctgatgtactgacaagcctcgcgtacccgattatccatc
ggtggatggagcgactcgttaatcgcttccatgcgccgcagtaacaatt
gctcaagcagatttatcgccagcagctccgaatagcgcccttccccttg
cccggcgttaatgatttgcccaaacaggtcgctgaaatgcggctggtgc
gcttcatccgggcgaaagaaccccgtattggcaaatattgacggccagt
taagccattcatgccagtaggcgcgcggacgaaagtaaacccactggtg
ataccattcgcgagcctccggatgacgaccgtagtgatgaatctctcct
ggcgggaacagcaaaatatcacccggtcggcaaacaaattctcgtccct
tgatttttcaccaccccctgaccgcgaatggtgagatgagaatataacc
tttcattcccagcggtcggtcgataaaaaaatcgagataaccgttggcc
tcaatcggcgttaaacccgccaccagatgggcattaaacgagtatcccg
gcagcaggggatcattttgcgcttcagccatacttttcatactcccgcc
attcagag

What is claimed is:

1. A polymerase-nucleic acid complex, said polymerase-nucleic acid complex comprising:
a target nucleic acid and a nucleic acid polymerase, wherein said polymerase has an attachment complex comprising at least one anchor, which said at least one anchor irreversibly associates said target nucleic acid with said polymerase to increase the processivity index.

2. The polymerase-nucleic complex of claim 1, wherein said polymerase-nucleic acid complex further comprises a primer nucleic acid which complements a region of said target nucleic acid.

3. The polymerase-nucleic complex of claim 1, wherein said attachment complex comprises at least two anchors.

4. The polymerase-nucleic complex of claim 3, wherein said attachment complex is attached to a support.

5. The polymerase-nucleic complex of claim 1, wherein said attachment complex comprises a topological tether.

6. The polymerase-nucleic complex of claim 3, wherein said at least two anchors further comprises a topological tether.

7. The polymerase-nucleic complex of claim 6, wherein said topological tether is attached to at least one anchor via a complementary binding pair.

8. The polymerase-nucleic complex of claim 6, wherein said topological tether is attached to at least two anchors via at least two complementary binding pairs.

9. The polymerase-nucleic complex of claim 7, wherein said complementary binding pairs are selected from the group consisting of any haptenic or antigenic compound in combination with a corresponding antibody or binding portion or fragment thereof, nonimmunological binding pairs, receptor-receptor agonist or antagonist, IgG-protein A, lectin-carbohydrate, enzyme-enzyme cofactor, enzyme-enzyme-inhibitor, and complementary polynucleotide pairs capable of forming nucleic acid duplexes.

10. The polymerase-nucleic complex of claim 9, wherein said complementary binding pair is selected from the group consisting of digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin, biotin-avidin, biotin-streptavidin, thyroxine and cortisol, a phenylalanine derivative and hydrazine linker and acetylcholine and receptor-acetylcholine.

11. The polymerase-nucleic complex of claim 1, wherein said at least one anchor comprises at least one amino acid or an epitope for attachment.

12. The polymerase-nucleic complex of claim 11, wherein said at least one amino acid is selected from the group consisting of a cysteine, a phenylalanine derivative and a histidine.

13. The polymerase-nucleic complex of claim 12, wherein said histidine is selected from the group consisting of a histidine tag, a histidine patch and a polyhistidine sequence.

14. The polymerase-nucleic complex of claim 5, wherein said topological tether comprises an antibody.

15. The polymerase-nucleic complex of claim 1, wherein said at least one anchor is attached to a support.

16. The polymerase-nucleic complex of claim 1, wherein said at least one anchor entraps said target nucleic acid.

17. The polymerase-nucleic complex of claim 6, wherein said topological tether is an antibody and said at least two anchors are each a histidine tag.

18. The polymerase-nucleic complex of claim 1, wherein said target nucleic acid is a circular DNA.

19. The polymerase-nucleic complex of claim 18, wherein said circular DNA is sequenced by strand displacement synthesis.

20. The polymerase-nucleic complex of claim 1, wherein said polymerase is a selected from a Family A polymerase and a Family B polymerase.

21. The polymerase-nucleic complex of claim 20, wherein said Family A polymerase is selected from the group consisting of Klenow, Taq, and T7 polyermase.

22. The polymerase-nucleic complex of claim 20, wherein said Family B polymerase is selected from the group consisting of a therminator polymerase, phi29, RB-69 and T4 polymerase.

23. The polymerase-nucleic complex of claim 1, wherein said polymerase-nucleic acid complex is an array of polymerase-nucleic acid complexes attached to a support.

24. The polymerase-nucleic complex of claim 23, wherein the plurality of members of said array of polymerase-nucleic acid complexes is randomly attached to said support.

25. The polymerase-nucleic complex of claim 23, wherein the plurality of members of said array of polymerase-nucleic acid complexes is uniformly attached to said support.

26. The polymerase-nucleic complex of claim 1, wherein the processivity index is at least 0.5.

27. The polymerase-nucleic complex of claim 26, wherein the processivity index is at least 0.8.

28. The polymerase-nucleic complex of claim 27, wherein the processivity index is 1.

29. A method for detecting incorporation of at least one NTP into a single primer nucleic acid molecule, said method comprising:
i. immobilizing onto a support a polymerase nucleic acid complex comprising a target nucleic acid, a primer nucleic acid which complements a region of the target nucleic acid, and at least one nucleic acid polymerase;
ii. contacting said immobilized complex with at least one type of labeled nucleotide triphosphate [NTP], wherein each NTP is labeled with a detectable label, and
iii. detecting the incorporation of said at least one type of labeled NTP into a single molecule of said primer, while said at least one type of labeled NTP is in contact with said immobilized complex, by detecting the label of the NTP while said at least one type of labeled NTP is in contact with said polymerase nucleic acid complex.

30. The method of claim 29, wherein said polymerase nucleic acid complex is contacted with a single type of labeled NTP.

31. The method of claim 29, wherein said polymerase nucleic acid complex is contacted with at least two different types of NTPs, and wherein each type of NTP is uniquely labeled.

32. The method of claim 29, wherein said polymerase nucleic acid complex is contacted with at least four different types of NTPs, and wherein each type of NTP is uniquely labeled.

33. The method of claim 29, wherein said NTPs are labeled on the γ-phosphate.

34. The method of claim 33, wherein said NTPs are labeled on the γ-phosphate with a fluorescent label.

35. The method of claim 29, wherein the detecting comprises detecting a unique signal from the labeled NTP using a system or device selected from the group consisting of an optical reader, a high-efficiency photon detection system, a photodiode, a camera, a charge couple device, an intensified charge couple device, a near-field scanning microscope, a far-field confocal microscope, a microscope that detects wide-field epi-illumination, evanescent wave excitation and a total internal reflection fluorescence microscope.

36. The method of claim 29, wherein the label of the NTP is detected using a method comprising a four color evanescent wave excitation device.

37. The method of claim 29, wherein said detecting is carried out by a mechanism selected from the group consisting of fluorescence resonance energy transfer, an electron transfer mechanism, an excited-state lifetime mechanism and a ground-state complex quenching mechanism.

38. The method of claim 29, wherein said detecting step comprises measuring a residence time of a labeled NTP in said polymerase nucleic acid complex.

39. The method of claim 38, wherein the residence time of an NTP that is incorporated into the primer nucleic acid is at least about 100 times longer to about 10,000 times longer than the residence time of an NTP that is not incorporated.

40. The method of claim 39, wherein the residence time of an NTP that is incorporated into the primer nucleic acid is at least about 200 times longer to about 500 times longer than the residence time of an NTP that is not incorporated.

41. The method of claim 38, wherein the residence time of an NTP that is incorporated into the primer nucleic acid is about 1.0 milliseconds to about 100 milliseconds.

42. The method of claim 41, wherein the residence time of an NTP that is incorporated into the primer nucleic acid is about 2.0 milliseconds to about 10 milliseconds.

43. The method of claim 29, further comprising the step of genotyping said target nucleic acid by determining the identity of at least one NTP that is incorporated into a single molecule of the primer.

44. The method of claim 29, further comprising: sequencing said target nucleic acid by determining the identity and sequence of incorporation of NTPs that are incorporated into a single molecule of the primer.

45. The method of claim 29, wherein said detection is a sequential detection of the identities of more than one uniquely labeled dNTPs that are sequentially incorporated into the primer, wherein said sequential detection yields the sequence of region of the target DNA that is downstream of the elongating end of the primer.

46. The method of claim 29, wherein said polymerase-nucleic acid complex comprises a target nucleic acid and a nucleic acid polymerase, wherein said polymerase has an attachment complex comprising at least one anchor, which irreversibly associates said target nucleic acid with said polymerase for increasing the processivity index.


SEQUENCE LISTING (45 sequences)

SEQ ID NO: 1 - DNA, 6379 bp, Artificial Sequence
  synthetic pBAD-HisC plasmid expression vector construct with modified 9 Degees North DNA polymerase (Therminator DNA polymerase) with AviTag leg insertions
aagaaaccaattgtccatattgcatcagacattgccgtcactgcgtcttttactgcctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattacatatgattctcgataccgactacatcaccgagaacgggaagcccgtgataagggtcttcaagaaggagaacggcgagtttaaaatcgagtacgacagaaccttcgagccctacttctacgcccttctgaaggacgattctgcgatagaggacgtcaagaaggtaaccgcaaagaggcacggaacggttgtcaaggtgaagcgcgccgagaaggtgcagaagaagttcctcggcaggccgatagaggtctggaagctctacttcaaccatcctcaggacgtcccggcgattcgagacaggatacgcgcccaccccgctgtcgttgacatctacgagtacgacatacccttcgccaagcgctacctcatcgacaagggcctgattccgatggagggcgacgaggagcttacgatgctcgccttcgcgatcgcaaccctctatcacgagggcgaggagttcggaaccgggccgattctcatgataagctacgccgacgggagcgaggcgagggtgataacctggaagaagattgaccttccgtacgttgacgtcgtctcgaccgagaaggagatgattaagcgcttcctccgcgtcgtcagggagaaggaccccgacgtgctcatcacctacaacggcgacaacttcgacttcgcctacctgaagaagcgctgtgaggaactcggaataaagttcacactcggcagggacgggagcgagccgaagatacagcgaatgggcgaccgctttgccgttgaggtgaagggcaggattcacttcgacctctaccccgtcataaggcgcacgataaacctcccgacctacacccttgaggccgtttacgaggccgtctttggaaagcccaaggagaaggtttacgcagaggagatagcgcaggcctgggagagcggggagggccttgaaagggttgcaagatactcgatggaggacgctaaggtgacctacgagctgggaagggagttcttcccgatggaggcccagctttcgaggcttataggccagagcctctgggacgtctcgcgctcgagcaccggaaatttggtggaggcattcctcctgcggaaggcctacaagaggaacgagctcgccccaaacaagcccgacgagagggagctcgcgagacggcgcgggggctacgctggcgggtacgttaaggaaccagagcggggattgtgggacaacattgtgtatttagacttccgctcgtggtatccttcaatcatcataacccacaacgtctcgccggataccctcaaccgcgagggctgtaaagagtacgacgtcgcccctgaggttggacacaagttctgcaaggacttccccggcttcataccaagcctcctgggagatttgctcgaggaggcgagcaagatagagcggaagatgaaggcaacggttgacccgctggagaagaaactcctcgtgtacaggcagtggcttataaaaatcctcgccaacagcttctacggctactacggctacgccaaggcccggtggtactgcaaggagtgcgccgagagcgttacggcctggggaagggagtatatagaaatggttatccgggaactcgaagaaaaattcggttttaaagttctctatgccgatacagacggtctccatgctaccattcccggagcagacgctgaaacagtcaagaaaaaagcaaaggagttcttaaaatacattaatccaaaactgcccggcctgctcgaacttgagtacgagggcttctacgtgaggggcttcttcgtcacgaagaagaagtacgctgtgatagacgaggagggcaagataaccacgaggggtcttgagattgtgaggcgcgactggagcgagatagcgaaggagacccaggccagggtcttagaggcgatactcaagcacggtgacgtcgaggaggccgttaggatagtcaaggaagtgacggaaaagctgagcaagtatgaggtcccgcccgagaagctggtaatccacgagcagataacgcgcgatttgagggattacaaagccaccggcccgcacgttgccgttgcgaagaggctcgcggcgcgtggagtgaaaatccggcccggcacggtgataagctacatcgtcctaaagggctctggaaggataggcgacagggcgattccagctgatgagttcgacccgacgaagcaccgctacgatgcggaatactacatcgagaaccaggttctcccggcggtggagaggattctaaaagccttcggctatcggaaggaggatttgcgctaccagaagacgaagcaggtcggctcgggcgcgtggctgaaggtgaaggggaagaagggtaccgaagcttacgtagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaatagcgcctgatgcggtattttctccttacgcatctgtgcggtatttcacaccgcatctggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 2 - DNA, 87 bp, Artificial Sequence
  synthetic nucleotide sequence sequenced by multiple sequencing embodiment
tatgaaaattttccggtttaaggcgtttccgwtcttcttcgtcataacttaatgtttttatttaaataccctctraaaagaaaggaa

SEQ ID NO: 3 - DNA, 87 bp, Artificial Sequence
  synthetic nucleotide sequence sequenced by multiple sequencing embodiment
cgacaggtgctgaaagcgaggctttttggcckctgtcgtttcctttctctgtttttgtccgtgaatgaacaatgraagtcaacaaaa

SEQ ID NO: 4 - DNA, 87 bp, Artificial Sequence
  synthetic nucleotide sequence sequenced by multiple sequencing embodiment
gcagctggctgacattttcggtgcgagtatcsgtaccattcagaactggcaggaacagggaatcccgttctgcgrggcggtggcaag

SEQ ID NO: 5 - DNA, 87 bp, Artificial Sequence
  synthetic nucleotide sequence sequenced by multiple sequencing embodiment
gtaatgaggtgctttatgactctgccgccgtyataaaatggtatgccgaaagggatgctgaaatgagaacgaaargctgcgccggga

SEQ ID NO: 6 - protein, 21 aa, Artificial Sequence
  synthetic Therminator DNA polymerase anchor insert at positions K53 and K229
LLSKKRSLCCXCTVIVYVTDT

SEQ ID NO: 7 - DNA, 25 bp, Artificial Sequence
  synthetic double-stranded oligonucleotide First adaptor
cgccacattacacttcctaacacgt

SEQ ID NO: 8 - DNA, 24 bp, Artificial Sequence
  synthetic double-stranded oligonucleotide First adaptor
cgtgttaggaagtgtaatgtggcg

SEQ ID NO: 9 - DNA, 25 bp, Artificial Sequence
  synthetic double-stranded oligonucleotide Second adaptor
cagtaggtagtcaaggctagagtct

SEQ ID NO: 10 - DNA, 24 bp, Artificial Sequence
  synthetic double-stranded oligonucleotide Second adaptor
gactctagccttgactacctactg

SEQ ID NO: 11 - DNA, 30 bp, Artificial Sequence
  synthetic portion of ligated DNA Product 1 and 2
cgccacattacacttcctaacacgtnnnnn

SEQ ID NO: 12 - DNA, 33 bp, Artificial Sequence
  synthetic portion of ligated DNA Product 1 and 3
nnnnnagactctagccttgactacctactgaaa

SEQ ID NO: 13 - DNA, 30 bp, Artificial Sequence
  synthetic portion of ligated DNA Product 1 and 3
cagtaggtagtcaaggctagagtctnnnnn

SEQ ID NO: 14 - DNA, 30 bp, Artificial Sequence
  synthetic portion of ligated DNA Product 2
nnnnnacgtgttaggaagtgtaatgtggcg

SEQ ID NO: 15 - DNA, 30 bp, Artificial Sequence; identical to SEQ ID NO: 11
  synthetic portion of ligated DNA Product 2

SEQ ID NO: 16 - DNA, 30 bp, Artificial Sequence; identical to SEQ ID NO: 13
  synthetic portion of ligated DNA Product 3

SEQ ID NO: 17 - DNA, 33 bp, Artificial Sequence; identical to SEQ ID NO: 12
  synthetic double-stranded oligonucleotide First adaptor

SEQ ID NO: 18 - DNA, 59 bp, Artificial Sequence
  synthetic primed circular template strand
nnnnncgtgttaggaagtgtaatgtggcgcagtaggtagtcaaggctagagtctnnnnn

SEQ ID NO: 19 - DNA, 49 bp, Artificial Sequence
  synthetic primed circular template primer
agactctagccttgactacctactgcgccacattacacttcctaacacg

SEQ ID NO: 20 - DNA, 27 bp, Artificial Sequence
  synthetic amplification forward primer for T7 DNA polymerase gene
atgatcgtttctgccatcgcagctaac

SEQ ID NO: 21 - DNA, 16 bp, Artificial Sequence
  synthetic amplification reverse primer for T7 DNA polymerase gene
tcagtggcaaatcgcc

SEQ ID NO: 22 - DNA, 75 bp, Artificial Sequence
  synthetic oligonucleotide encoding Strep-Tag II sequence overlapping 5′-end of amplified T7 exo-polymerase gene
atgtccaactggtcccacccgcagttcgaaaaaggtggaggttccgctatgatcgtttctgccatcgcagctaac

SEQ ID NO: 23 - protein, 25 aa, Artificial Sequence
  synthetic peptide with Strep-Tag II peptide, spacer and T7 polymerase N-terminus overlap
MSNWSHPQFEKGGGSAMIVSAIAAN

SEQ ID NO: 24 - DNA, 20 bp, Artificial Sequence
  synthetic overlapping PCR StrepTag forward primer
atgtccaactggtcccaccc

SEQ ID NO: 25 - DNA, 16 bp, Artificial Sequence
  synthetic overlapping PCR StrepTag reverse primer
tcagtggcaaatcgcc

SEQ ID NO: 26 - DNA, 21 bp, Artificial Sequence
  synthetic oligonucleotide primer derived from cystic fibrosis transmembrane conductance regulator gene normal allele and (delta)F508 deletion mutant
tactataaaagaaattaccac

SEQ ID NO: 27 - RNA, 22 nt, Artificial Sequence
  synthetic oligonucleotide template derived from cystic fibrosis transmembrane conductance regulator gene normal allele
gugguaauuucuuuuauaguag

SEQ ID NO: 28 - RNA, 22 nt, Artificial Sequence
  synthetic oligonucleotide template derived from cystic fibrosis transmembrane conductance regulator gene (delta)F508 deletion mutant
gugguaauuucuuuuauaguaa

SEQ ID NO: 29 - protein, 21 aa, Artificial Sequence
  synthetic AviTag peptide substrate for E. coli biotin-protein ligase flanked by arbitrarily-chosen amino acids
SSGLNDIFEAQKIEWHEGASS

SEQ ID NO: 30 - DNA, 28 bp, Artificial Sequence
  synthetic unlabeled PCR primer for M13 DNA
cgcctgcaacagtgccacgctgagagcc

SEQ ID NO: 31 - DNA, 28 bp, Artificial Sequence
  synthetic 5′-labeled PCR primer for M13 DNA
cacgacgttgtaaaacgacggccagtgc

SEQ ID NO: 32 - protein, 6 aa, Artificial Sequence
  synthetic 6xHis tag
HHHHHH

SEQ ID NO: 33 - DNA, 18 bp, Artificial Sequence
  synthetic (CAT)-6 encoding 6xHis tag
catcatcatcatcatcat

SEQ ID NO: 34 - DNA, 86 bp, Artificial Sequence
  synthetic PCR primer p1 for constucting K53-V54 AviTag leg insertion
gctagatgcgccttcgtgccattcgattttctgagcttcgaagatgtcgttcagaccgctagacttcttgacgtcctctatcgcag

SEQ ID NO: 35 - DNA, 23 bp, Artificial Sequence
  synthetic PCR primer p2 for constucting K53-V54 AviTag leg insertion
cttcttgacgtcctctatcgcag

SEQ ID NO: 36 - DNA, 83 bp, Artificial Sequence
  synthetic PCR primer p3 for constucting K53-V54 AviTag leg insertion
tctagcggtctgaacgacatcttcgaagctcagaaaatcgaatggcacgaaggcgcatctagcgtaaccgcaaagaggcacgg

SEQ ID NO: 37 - DNA, 20 bp, Artificial Sequence
  synthetic PCR primer p4 for constucting K53-V54 AviTag leg insertion
gtaaccgcaaagaggcacgg

SEQ ID NO: 38 - DNA, 22 bp, Artificial Sequence
  synthetic ampicillin resistance diagnostic amplification primer
ccttctgaaggacgattctgcg

SEQ ID NO: 39 - DNA, 19 bp, Artificial Sequence
  synthetic ampicillin resistance diagnostic amplification primer
cgcttcaccttgacaaccg

SEQ ID NO: 40 - DNA, 87 bp, Artificial Sequence
  synthetic PCR primer p1 for constucting second K53-V54 AviTag leg insertion
gctagatgcgccttcgtgccattcgattttctgagcttcgaagatgtcgttcagaccgctagactttattccgagttcctcacagcg

SEQ ID NO: 41 - DNA, 24 bp, Artificial Sequence
  synthetic PCR primer p2 for constucting second K53-V54 AviTag leg insertion
ctttattccgagttcctcacagcg

SEQ ID NO: 42 - DNA, 83 bp, Artificial Sequence
  synthetic PCR primer p3 for constucting second K53-V54 AviTag leg insertion
tctagcggtctgaacgacatcttcgaagctcagaaaatcgaatggcacgaaggcgcatctagcttcacactcggcagggacgg

SEQ ID NO: 43 - DNA, 20 bp, Artificial Sequence
  synthetic PCR primer p4 for constucting second K53-V54 AviTag leg insertion
ttcacactcggcagggacgg

SEQ ID NO: 44 - DNA, 20 bp, Artificial Sequence
  synthetic ampicillin resistance diagnostic amplification primer
ttcgctgtatcttcggctcg

SEQ ID NO: 45 - DNA, 21 bp, Artificial Sequence
  synthetic ampicillin resistance diagnostic amplification primer
tacctgaagaagcgctgtgag